SEQUENCE LISTING (36 sequences)

SEQ ID NO: 1 - DNA, 24 bp, Mus musculus
gaattctgcaggatgggggcttct

SEQ ID NO: 2 - DNA, 24 bp, Mus musculus
ggatccttattgccagcaagttgt

SEQ ID NO: 3 - DNA, 26 bp, Mus musculus
gcctcgaggaaactatgctttggaga

SEQ ID NO: 4 - DNA, 81 bp, Mus musculus
gcgctcagcttaacgatgattccacaccattttctgtgcatccagaatatgatgcaggccatagcaaggctctgaaatttg

SEQ ID NO: 5 - DNA, 26 bp, Mus musculus
gggaattcactaaaatgtcaacctct

SEQ ID NO: 6 - DNA, 23 bp, Mus musculus
gggaattccttgggatgctgctc

SEQ ID NO: 7 - DNA, 26 bp, Mus musculus
ccggatccttatttcatgcaatcttc

SEQ ID NO: 8 - DNA, 31 bp, Mus musculus
ctcgagatgcctcctcacagcctgttctgtg

SEQ ID NO: 9 - DNA, 26 bp, Mus musculus
gaattccagaccatgaagaaccctgg

SEQ ID NO: 10 - DNA, 26 bp, Mus musculus
ctcgagggaaggatgattcttgccgc

SEQ ID NO: 11 - DNA, 517 bp, Mus musculus
ccttgggatgctgctcctgagatggcccaccttctgctgcctctgggtttgtaagaggacatttctgggtgaactcatcctcatttccatttcttcaaatactgttttctttgctcatgagaacttttccctttgcttgcagttgggcttgggcagctggagcaaactgaattatcggtcaccagagagacagatgagagtgcgcaaatatcctgtatagtttctcttccatatttctccaacacagctatacattggtaccggcaaaaagcaaaaaagtttgagtatctaatatatgtctcaacaaactacaatcaacgacccttaggagggaagaacaaaaaaattgaagcaagtaaagattttcaaacttctacctcaaccttgaaaataaattacttgaagaaagaagatgaagctacctactactgtgcagtctggataaacacaacattagagcctctagactagcctgcataagaaccatccccatctgtgcccacccacatcctcttgg

SEQ ID NO: 12 - protein, 115 aa, Mus musculus
MLLLRWPTFCCLWVFGLGQLEQTELSVTRETDESAQISCIVSLPYFSNTAIHWYRQKAKKFEYLIYVSTNYNQRPLGGKNKKIEASKDFQTSTSTLKINYLKKEDEATYYCAVWI

SEQ ID NO: 13 - DNA, 390 bp, Mus musculus
ccgtccacattgctgtttcttcccctgccagtcattcatgggtgtttttctcgtttgcttgcagttggacatgggaagttggagcaacctgaaatatcaatttccagaccaagagatgagactgcacaaatatcctgtaaagttttcatcgaaagctttaggagtgtaaccatacactggtaccggcagaaaccaaaccaaggtttagagtttctattatatgtccttgcaacccctacccatattttcttagataaggagtacaagaaaatggaggcaagtaaaaatcctagtgcttctacatcgatattgacaatatattccttggaggaagaagacgaagctatctactactgttcctacggctatagctcaggttttcacaaggta

SEQ ID NO: 14 - protein, 146 aa, Mus musculus
MKNPGSQALLPLYLPWEANLADENPLLKVVIFLCLLTFGHGKLEQPEISISRPRDETAQISCKVFIESFRSVTIHWYRQKPNQGLEFLLYVLATPTHIFLDKEYKKMEASKNPSASTSILTIYSLEEEDEAIYYCSYGYSSGFHKV

SEQ ID NO: 15 - DNA, 477 bp, Mus musculus
agccactaaaatgtcaacctcttggctttttcttctgtctttgacctgtgtttgtaagttaattttgcttcttttttccctttcgttatttttgtgtcctgagataactgggaataaaaatgattgggaaaatctctgatgtctcttccttgctttccagatggagactcctggatatctcaggatcagctctcctttacccgaagaccaaacaagacggtgcacataagttgcaagctctctggggttccccttcataacaccattgtgcactggtaccaactgaaagaaggggagcccctgagacgaatcttctatggctcagtcaaaacttacaaacaagacaagtcccactcccgcttggaaattgatgagaaggatgatggtaccttttacctgataatcaacaatgttgtcacatcggatgaagccacgtactactgtgcctgctgggatagctcaggttttcacaaggta

SEQ ID NO: 16 - protein, 120 aa, Mus musculus
MSTSWLFLLSLTCVYGDSWISQDQLSFTRRPNKTVHISCKLSGVPLHNTIVHWYQLKEGEPLRRIFYGSVKTYKQDKSHSRLEIDEKDDGTFYLIINNVVTSDEATYYCACWDSSGFHKV

SEQ ID NO: 17 - DNA, 336 bp, Mus musculus
tcagagggaacgagtctcacgtcacctctggggtcatatgtcatcaagaggaaaggaaatacggcttttctcaaatgtcaaataaaaacaagtgttcagaagcccgatgcatacatacactggtaccaagagaagccaggccagcgtctccaaagaatgctgtgtagttcttcaaaagaaacaattgtctatgagaaagattttagtgacgaaagatatgaggcaaggacatggcagagtgatttgtcttcagtcctcaccatacaccaagtgacagaagaggacacgggaacttattactgtgcatgctgggatagctcaggttttcacaaggta

SEQ ID NO: 18 - protein, 112 aa, Mus musculus
SEGTSLTSPLGSYVIKRKGNTAFLKCQIKTSVQKPDAYIHWYQEKPGQRLQRMLCSSSKETIVYEKDFSDERYEARTWQSDLSSVLTIHQVTEEDTGTYYCACWDSSGFHKV

SEQ ID NO: 19 - DNA, 365 bp, Mus musculus
gttcttatagcacaaggcatgctgtgggctctggccctacttctagctttcttgccggctggcagacaaacatcctccaacttggaagaaagaataatgtcaatcaccaagctagaggggtcctctgctataatgacttgtgatactcacagaacaggcacttacatccactggtaccgattccagaaagggagggccccagagcaccttctctactataacttcgtcagttccacaactgtggtggattccagattcaatttggagaaatatcatgtttatgaaggcccggacaagaggtataaatttgtgcttcggaatgtggaggagtccgattctgctctgtactactgtgcctcctgggc

SEQ ID NO: 20 - protein, 121 aa, Mus musculus
VLIAQGMLWALALLLAFLPAGRQTSSNLEERIMSITKLEGSSAIMTCDTHRTGTYIHWYRFQKGRAPEHLLYYNFVSSTTVVDSRFNLEKYHVYEGPDKRYKFVLRNVEESDSALYYCASW

SEQ ID NO: 21 - DNA, 466 bp, Mus musculus
gaatttcgctcacctcagtgaaactatgctttggagatgtccagtcctctgtatattcatcttcagtacagggacctctttggatgtatatttggaaccagttgccaaaacttttactgttgtagctggggatcctgcctccttctactgcactgtaacaggaggggacatgaagaattatcatatgagctggtataagaagaatggaactaatgctctgtttttagtatacaagctaaatagcaattctactgatggtggaaagagcaacctcaaagggaaaattaacatttcaaaaaatcagtttatactcgacattcagaaggcaacaatgaaagacgctggtacgtactactgtgggtcagatatcggagggagctcctgggacacccgacagatgttttttggaactggcatagagctctttgtggagccccaaagccagcctccggccaaaccatctgtt

SEQ ID NO: 22 - protein, 140 aa, Mus musculus
MLWRCPVLCIFIFSTGTSLDVYLEPVAKTFTVVAGDPASFYCTVTGGDMKNYHMSWYKKNGTNALFLVYKLNSNSTDGGKSNLKGKINISKNQFILDIQKATMKDAGTYYCGSDIGGSSWDTRQMFFGTGIELFVEPQSQ

SEQ ID NO: 23 - DNA, 431 bp, Mus musculus
ctatcttctgttgagtcctctgagctggtcagtgtctgggatgcagacgctactatggcctcctttcctcttcacagacaaggatgtgctgtgcatcacgctgacccagagctccactgaccagacagtggcaagcggcactgaagtaacactgctttgcacgtacaatgcggattctccaaacccagatttattttggtatcgcaaaaggccagacagatccttccagttcatcctttatagggacgacactagttcccatgatgcagattttgttcaaggtcgattttctgtgaagcacagcaaggccaacagaaccttccatctggtgatctctccagtgagccttgaagacagcgctacttattactgtgcctcggggtatcggcggatacgagccctcgctaccgacaaactcgtctttggacaag

SEQ ID NO: 24 - protein, 130 aa, Mus musculus
MQTLLWPPFLFTDKDVLCITLTQSSTDQTVASGTEVTLLCTYNADSPNPDLFWYRKRPDRSFQFILYRDDTSSHDADFVQGRFSVKHSKANRTFHLVISPVSLEDSATYYCASGYRRIRALATDKLVFGQ

SEQ ID NO: 25 - DNA, 427 bp, Mus musculus
atgcctcctcacagcctgttctgtgtgctggtggccttgcgtttctctggatctaatgtggccgagaaagtgattcaggtctggtcaacagcaagcaggcaggagggcgaagaactcaccctggactgttcatatgagacaagtcaggtcttataccatcttttctggtacaagcaccttcttagtggagagatggttttccttattcgacaaacgtcttcttctactgcaaaagagaggagcggccgctattctgtagtcttccagaaatcactcaaatccatcagccttatcatttcagctttacaaccagacgattcgggaaagtatttctgtgctctctgggagctccaatcaaggagggtctgcgaagctcatctttggggaggggacaaagctgacagtgagctcatacatccagaaccca

SEQ ID NO: 26 - protein, 142 aa, Mus musculus
MPPHSLFCVLVALRFSGSNVAEKVIQVWSTASRQEGEELTLDCSYETSQVLYHLFWYKHLLSGEMVFLIRQTSSSTAKERSGRYSVVFQKSLKSISLIISALQPDDSGKYFCALWELQSRRVCEAHLWGGDKADSELIHPEP

SEQ ID NO: 27 - DNA, 518 bp, Homo sapiens
atgctgttggctctagctctgcttctagctttcctgcctcctggtaagagtgctgcctacagagaggctcacaggttttattttgtttcgttttgtttattttcttcttttgcaagggataccatactaagaaatgcctcattacattttgtgttgttcccattgcagccagtcagaaatcttccaacttggaagggagaacaaagtcagtcaccaggccaactgggtcatcagctgtaatcacttgtgatcttcctgtagaaaatgccgtctacacccactggtacctacaccaggaggggaaggccccacagcgtcttctgtactatgactcctacaactccagggttgtgttggaatcaggaatcagtcgagaaaagtatcatacttatgcaagcacagggaagagccttaaatttatactggaaaatctaattgaacgtgactctggggtctattactgtgccacctgggataggcacagtgattcagacctgtgctacaccacactgaaaatc

SEQ ID NO: 28 - protein, 118 aa, Homo sapiens
MLLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDR

SEQ ID NO: 29 - DNA, 825 bp, Homo sapiens
atgctgtcactgctccacgcatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggcccagcaagagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattgggaagaaaagaagagcaacacgattctgggatcccaggaggggaacaccatgaagactaatgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagtaa

SEQ ID NO: 30 - protein, 274 aa, Homo sapiens [sequence omitted]

SEQ ID NO: 31 - DNA, 783 bp, Homo sapiens
atgcagaggatctcctccctcatccatctctctctcttctgggcaggagtcatgtcagccattgagttggtgcctgaacaccaaacagtgcctgtgtcaataggggtccctgccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacaccttggggatggggggggaatacaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaagtaa

SEQ ID NO: 32 - protein, 260 aa, Homo sapiens [sequence omitted]

SEQ ID NO: 33 - DNA, 720 bp, Homo sapiens
tcctcttgttttcctcccgtcttctttcattagaaccgtcagatagtctctgagatcttaggaaccctgttttgttgtatatcctctgccctgatatgaagaaaagatgattcttactgtgggctttagctttttgtttttctgtaagtagtttcatttgtttacattccacttgctctataaccccatttctccttctctgactcttgaaggctttttctcttgcactcttggtttttggttatttgctgcctttggctttatttctgtgtccttctcttgcctctgctcattgagttcttctgggctaggcatgtgctgtactcactgtctggggggtgcagaattcactatttcctcattgtctttttcccagacaggggcacgctgtgtgacaaagtaacccagagttccccggaccagacggtggcgagtggcagtgaggtggtactgctctgcacttacgacactgtatattcaaatccagatttattctggtaccggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctttagcactatgatgcaggtgcccaggaagtcataacacaaactc

SEQ ID NO: 34 - protein, 113 aa, Homo sapiens
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCAF

SEQ ID NO: 35 - DNA, 1186 bp, Homo sapiens
gttcacttcacagtacagagtcctgaaaataaagaagaaaatttttttttatctagaaaaagaaccaaacatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtatttctgtgcaatgagagcggtgtacaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttgtaaggctgactggcatgaggaagctacactcctgaagaaaccaaaggcttacaaaaatgcatctccttggcttctgacttctttgtgattcaagttgacctgtcatagccttgttaaaatggctgctagccaaaccactttttcttcaaagacaacaaacccagctcatcctccagcttgatgggaagacaaaagtcctggggaaggggggtttatgtcctaactgctttgtatgctgttttataaagggat

SEQ ID NO: 36 - protein, 288 aa, Homo sapiens [sequence omitted]

(19) United States
(12) Patent Application Publication
(10) Pub. No.: US 2007/0269426 A1
(43) Pub. Date: Nov. 22, 2007

(54) Use of Soluble Gammadelta Cell Receptors for Regulating T Cell Function

(75) Inventors: Rebecca L. O'Brien, Denver, CO (US); Willi K. Born, Denver, CO (US); Christina L. Roark, Highlands Ranch, CO (US); M. Kemal Aydintug, Thornton, CO (US)

Correspondence Address: SHERIDAN ROSS PC, 1560 BROADWAY, SUITE 1200, DENVER, CO, 80202, US

(73) Assignee: NATIONAL JEWISH MEDICAL AND RESEARCH CENTER, Denver, CO (US)

(21) Appl. No.: 11/695,986

(22) Filed: Apr. 3, 2007

Related U.S. Application Data
(63) Continuation of application No. 10/340,536, filed on Jan. 10, 2003, now abandoned.
(60) Provisional application No. 60/347,285, filed on Jan. 10, 2002.

(51) Int. Cl. A61K 39/395

(52) U.S. Cl. 424/131.1

(57) ABSTRACT

Disclosed is a method of using soluble γδ T cell receptors to regulate a γδ T cell-mediated immune response in a mammal.

### CROSS-REFERENCE TO RELATED APPLICATIONS

This application is a Continuation of U.S. application Ser. No. 10/340,536, filed Jan. 10, 2003, which claims the benefit of priority under 35 U.S.C. §119(e) to U.S. Provisional Application Ser. No.60/347,285, filed Jan. 10, 2002. The entire disclosure of U.S. Provisional Application Ser. No. 60/347,285 is incorporated herein by reference.

### GOVERNMENT SUPPORT

This invention was made in part with government support under Grant Nos. R01AI44920, T32AI07405, R01AI40611, and R01HL65410, each awarded by the National Institutes of Health. The government has certain rights to this invention.

### FIELD OF THE INVENTION

The present invention relates to the use of soluble γδ T cell receptors for the treatment of specific diseases and/or conditions mediated by γδ T cells or their ligands.

### BACKGROUND OF THE INVENTION

Mammals are known to have two types of T cells: αβ T cells and γδ T cells. The role of αβ T cells is well-established in host protection against infectious agents, based on the ability of these cells to recognize proteins from foreign microbes via their T cell receptors, which, for each αβ T cell, is a unique molecule having its own specificity. These lymphocytes, as well as B lymphocytes, bear antigen receptors (i.e., αβ TcRs and B cell receptors) having the potential to recognize foreign molecules. Both types of lymphocytes also undergo developmental screening processes to ensure that they will not bind to self molecules and trigger an autoimmune attack. While the γδ T cells also bear cell surface TcRs, related to but distinct from those carried by αβ T cells, the types of molecules that γδ TcRs recognize has not yet been resolved.

However, many studies have now shown that γδ T cells can modulate inflammatory and adaptive immune responses, suggesting that these cells play an immunoregulatory role. Although abundant in many animals, the γδ T cells are relatively rare in the lymphoid organs of mice and humans, but are over-represented in certain epithelia, notably those of the skin, intestine, reproductive tract and lung, implying that they may participate in protection against agents that enter the body at interfaces with the physiologic exterior. Within these epithelial sites in both mice and humans, the T cell receptors carried by the γδ T cells differ. For this reason, and because their T cell receptors are more limited in structure than are αβ T cell receptors, γδ T cells are commonly regarded as different subsets based upon the Vγ and/or Vδ chains present in their T cell receptors. Recent evidence from the present inventors' laboratory indicates that these γδ T cell subsets also differ from one another functionally.

Considerable evidence indicates that instead of recognizing foreign molecules, at least some γδ T cells recognize autologous ligands produced by the host during infection or inflammation (Crowley et al., Science 287:314-316 (2000); Ezquerra et al., Eur J Immunol 22:491-498 (1992); Ferrick et al., Immunol Rev 120:51-69 (1991); Fisch et al.,1990, Science 250:1269-1273 (1990); Havran et al., Science 252:1430-1432 (1991); Mukasa et al., J Immunol 162:4910-4913 (1999); O'Brien et al., Cell 57:667-674 (1989); Wilde et al., Eur J Immunol 22:483-489 (1992)). These observations are consistent with other findings indicating that γδ T cells play a regulatory role during inflammation (Ferrick et al., Nature 373:255-257 (1995); Fu et al., J Immunol 153:3101-3115 (1994); Huber et al., J Virol 73 (1999); Lahn et al., Nature Medicine 5:1150-1156 (1999); Mombaerts et al., Nature 365:53-56 (1993); Suzuki et al., J Immunol 154:4476-4484 (1995); Yoshikai et al., J UOEH 15:246-254 (1993)). Controversies over the role they play may be explained by recent findings indicating that different γδ T cell subsets carry out distinct functions (Carding et al., J Exp Med 172:1225-1231 (1990); Huber et al., J Immunol 165:4174-4181 (2000); O'Brien et al., 2001, Chemical Immunology; O'Brien et al., J Immunol 165:6472-6479 (2000)). These γδ T cell subsets are defined by the expression of particular Vγs or Vγ/Vδ combinations in their TcR.

Because functional role and TCR expression are correlated in these subsets, it may seem logical to assume that the TCR must be engaged in order to elicit the functions of each subset. However, many T cell functions involve receptors other than the TCR (e.g., chemokine receptors). Moreover, recent reports of other receptors on γδ T cells that can activate cells (Bauer et al., Science 285:727-729 (1999); Hanby-Flarida et al., Immunology 88:116-123 (1996); Mokuno et al., J Immunol 165:931-940 (2000); Skeen et al., J Immunol 154:5832-5841 (1995); Takano et al., J Immunol 161:3019-3025 (1998)) have raised the possibility that the γδ TCR may function only during development or in “homing” and stand relatively inert in bringing about cellular function in mature cells. That in vivo γδ T cell responses generally involve entire subsets also adds credence to this argument, since antigen receptor junctions usually play the largest role in determining antigen specificity, and the TCR junctions within some γδ T cells responding as subsets can be quite diverse (O'Brien et al., Immunol. Rev. 121:155-170(1991); Ohmen et al., J. Immunol. 147:3353-3359 (1991)). Finally, self-reactivity among γδ T cells could also reflect responses mediated through receptors other than the TCR. Several lines of experimentation have shown that γδ T cell responses are more quickly and easily elicited in vitro than are αβ T cell responses, and it has been suggested that this is because signals through the TCR are not required (Leclercq et al., Scand J Immunol 36:833-841(1992); Skeen et al., J Exp Med 178:985-996 (1993); Lahn et al., J Immunol 160:5221-5230 (1998); Tough et al., J Exp Med 187:357-365 (1998)).

Therefore, prior to the present invention, the potential therapeutic benefits of targeting γδ T cells or their ligands was not clear.

### SUMMARY OF THE INVENTION

One embodiment of the present invention relates to a method to regulate a γδ T cell-mediated immune response in a mammal, comprising administering to the mammal a soluble γδ T cell receptor. The soluble γδ T cell receptor can include a single γ chain and a single δ chain linked by a disulfide bond in one aspect. In another aspect, the soluble γδ T cell receptor is a multimer of soluble γδ T-cell receptors comprising γ chains and δ chains linked by disulfide bonds. The soluble γδ T cell receptor can include any combination of Vγ and Vδ chains. In one aspect, the soluble γδ T cell receptor can comprise a murine Vγ chain chosen from a Vγ chain including, but not limited to: Vγ1, Vγ4, Vγ5, Vγ6, or Vγ7. In one aspect, the soluble γδ T cell receptor can comprise a murine δ chain chosen from a Vδ chain including, but not limited to: Vδ1, Vδ5, or Vδ6.3. In another aspect, the soluble γδ T cell receptor can comprise a human Vγ chain chosen from a Vγ chain including, but not limited to: Vγ8 or Vγ9. In another aspect, the soluble γδ T cell receptor can comprise a human Vδ1 chain.

The soluble γδ T cell receptor is, in one aspect, administered at a dose of from about 0.01 microgram×kilogram⁻¹ and about 10 milligram×kilogram⁻¹ body weight of the mammal. In another aspect, the soluble γδ T cell receptor is administered at a dose of from about 0.1 microgram×kilogram⁻¹ and about 10 milligram×kilogram⁻¹ body weight of the mammal. In one aspect, the step of administering the soluble γδ T-cell receptor is by a route selected from the group consisting of: aerosol, topical, intratracheal, transdermal, subcutaneous, intradermal, intravenous, intramuscular, intraperitoneal and direct injection to a tissue. In one embodiment, the mammal to be treated is a human.

The method of the present invention can be used to treat any disease or condition in which regulation of γδ T cells might be beneficial. In one aspect, the mammal has or is at risk of developing an intestinal condition (e.g., Crohn's disease, ischemic colitis, irritable bowel disease, or colon cancer). In another aspect, the mammal has or is at risk of developing a lung condition associated with inflammation (e.g., airway hyperresponsiveness, pneumonia, tuberculosis, and a primary or metastatic lung tumor). In another aspect, the mammal has or is at risk of developing a skin condition associated with inflammation (e.g., a skin lesion caused by bacterial infection, viral infection or laceration, and a skin cancer). In yet another aspect, the mammal has or is at risk of developing a condition associated with inflammation of the reproductive tract (e.g., infection caused by bacterial or viral infection that involve the epithelial mucosal lining, a tubal infection, preventing tubal factor infertility, or a cancer selected from the group consisting of ovarian cancer, cervical cancer, uterine cancer, prostate cancer or testicular cancer).

In another aspect, the mammal has or is at risk of developing inflammation caused by a γδ T cell subset, and wherein the soluble γδ T cell receptor is a soluble T cell receptor expressed by the γδ T cell subset. The soluble γδ T cell receptor can include a murine Vγ6 chain and a murine Vδ1 chain, a human Vγ8 or Vγ9 chain and a human Vδ2 chain, or the equivalent receptor thereof. In another aspect, the mammal has or is at risk of developing myocarditis caused by a γδ T cell subset, and wherein the soluble γδ T cell receptor is a soluble T cell receptor expressed by the γδ T cell subset. In this embodiment, the soluble γδ T cell receptor comprises a murine Vγ4 chain, a human Vγ9 chain, a human Vγ8 chain or the equivalent receptor thereof. In another aspect, the administration of the soluble γδ T cell receptor increases the activity of a γδ T cell subset expressing a murine Vγ1⁺ T cell receptor, a human Vγ9⁺ T cell receptor, or the equivalent receptor thereof. In another aspect, the mammal has or is at risk of developing an infection with Listeria monocytogenes, the soluble γδ T cell receptor comprises a murine Vγ1 chain, a murine Vγ6 chain, a human Vγ9 chain, a human Vγ8 chain, or the equivalent thereof, and administration of the soluble γδ T cell receptor increases clearance of Listeria monocytogenes from the mammal. In another aspect, the mammal has or is at risk of developing airway hyperresponsiveness caused by inflammation, the soluble γδ T cell receptor does not comprise a murine Vγ4 chain, a human Vγ9 chain, or the equivalent thereof, and administration of the soluble γδ T cell receptor results in an increase in the activity of a γδ T cell subset that expresses the murine Vγ4, the human Vγ9, or the equivalent thereof so that airway hyperresponsiveness is reduced in the mammal.

Yet another embodiment of the invention relates to a composition for regulating a γδ T cell-mediated immune response in a mammal, comprising: (a) a soluble γδ T cell receptor; and (b) an agent that regulates inflammation in the mammal.

### BRIEF DESCRIPTION OF THE FIGURES OF THE INVENTION

FIG. 1A is a diagram showing the competition assay used to assess soluble γδ T cell receptors.

FIGS. 1B-1D are flow cytometry graphs showing the retention of the ability of a sTcR to bind a monoclonal antibody as a reduction in the log fluorescence intensity; anti-Vγ5/Vδ1 (FIG. 1B), anti-Vγ1/Vδ6.3 (FIG. 1C), anti-αβ (FIG. 1D).

FIG. 2A is a series of bar graphs showing the percentage of Vγ1⁻/Vδ4⁻ γδ T cells in the liver during Listeria infection.

FIG. 2B is a series of bar graphs showing the percentage of Vγ1⁻/Vδ4⁻ γδ T cells in the spleen during Listeria infection.

FIG. 2C is a series of bar graphs showing the numbers of Vγ1⁻/Vδ4⁻ γδ T cells in the liver during Listeria infection.

FIG. 2D is a series of bar graphs showing Vγ1⁺ and Vγ4⁺ cell expansion in the liver during Listeria infection after treatment with sVγ5/Vδ1 or sTcR-αβ.

FIG. 3 is a bar graph showing the clearance of Listeria after treatment of mice with Vγ6/Vδ1 γδ sTCR.

### DETAILED DESCRIPTION OF THE INVENTION

The present invention generally relates to the use of soluble γδ T cell receptors to regulate the activity of γδ T cells or the ligands recognized by the T cell receptors. More specifically, the present invention relates to the use of various γδ T cell receptor subsets that have been generated in soluble form by an expression system and that are useful for treating various pathologies that are associated with the particular γδ T cell subset or its ligand(s). The targeted use of specific γδ T cell receptor subsets reduces the possibility of side effects seen with the more general treatments of these diseases. Virtually any route of administration and/or delivery vehicle suitable for delivery of the soluble receptor to a mammal is expressly encompassed in the present invention.

The present inventors have discovered that soluble γδ T cell receptors can be used to regulate inflammation in vivo, and that both expansion and immunoregulatory function of the γδ subsets appear to be dependent upon engagement of the TCR. To test whether the γδ TCR is necessary for the response of a γδ T cell subset during inflammation, the present inventors have carried out a series of experiments using a novel method, in which mice infected with bacteria were treated with a soluble version of the TCR of the mouse Vγ6/Vδ1⁺ subset. It was reasoned that if the responses of Vγ6/Vδ1⁺ γδ T cells require stimulation of their TCR via binding of a ligand, excess amounts of soluble TCR might out compete the normal TCR borne by these cells in ligand binding, and thus block Vγ6/Vδ1 cell activation. The Vγ6/Vδ1⁺ cells were chosen as a representative γδ T cell subset to examine for several reasons (note: the variable (V) region of the γ chain has a particular sequence which is known in the art as Vγ6, and the V region of δ chain has a particular sequence which is known in the art as Vδ1, following the nomenclature of Tonegawa et al. (Maeda et al., Proc Natl Acad Sci USA 84:6539-6540 (1987)). First, in an earlier study, the inventors had already established that an in vivo response of this subset could be elicited by inflammation alone, using a disease model in which no infectious agents or their products were introduced (Mukasa et al., J Immunol 162:4910-4913 (1999)). Thus, the Vγ6/Vδ1 subset would allow the testing of whether a subset response for which self-derived stimuli are known to be involved would require the TCR. Second, the Vγ6/Vδ1 subset also responds strongly during infection with Listeria, within the first few days of infection (Roark et al., J Immunol 156:2214-2220 (1996)), providing an easily detectable response in a short time, so that the soluble TCR need not persist for long periods. Third, the Vγ6/Vδ1 subset may be considered to be prototypical among γδ T cell subsets, because ˜90% of its constituents bear TcRs that are actually invariant (Asamow et al., Cell 55:837-847 (1988). TCR diversity among γδ T cells is in general considerably lower than its potential, as compared to αβ TcRs and BCRs, often as a result of developmental processes which give rise to certain Vγ/Vδ pair combinations. In the case of the Vγ6/Vδ1 subset, the developmental control extends to the TCR junctions as well, such that most Vγ6/Vδ1⁺ cells bear a “canonical” TCR, perhaps due to a need to ensure a particular γδ TCR specificity. Finally, the Vγ6/Vδ1 subset was chosen because this method could provide a way to directly examine the functional role of this subset. This was not possible before because of the lack of a monoclonal antibody specific for this subset, or of a genetically ablated mouse lacking only this subset. The results described herein demonstrate that both expansion and immunoregulatory function of the mouse Vγ6/Vδ1 subset are dependent upon engagement of the TCR.

In contrast, results from several laboratories over the past several years have suggested that for the γδ T lymphocytes (e.g., in contrast to αβ T lymphocytes), the TCR may be a non-critical component in the activation of these cells. First, γδ T cells have been shown in a number of different systems to influence events occurring early in an inflammatory or immune response, suggesting that their activation might in fact be driven by receptors such as those common on macrophages, neutrophils, or NK cells, which respond at early time points. In fact, the autologous responses found in some γδ T cell subsets could reflect activation via these non-TCR ligands, especially in cases such as the mouse Vγ1⁺ subset, in which a large degree of variation in the TCR γ and δ CDR3 regions is tolerated without loss of responsiveness. Second, the present inventors' findings that function and TCR type co-segregate among γδ T cells might be considered to be evidence that the TCR acts to direct processes other than peripheral activation, such as γδ T cell maturation or the homing to particular tissues. The mouse Vγ5/Vδ1 subset is perhaps one of the best candidates for this, since its TCR is certainly under tight developmental control (Sunaga et al., J Immunol 158:4223-4228 (1997); Zhang et al., Immunity 3:439-447 (1995)) and its distribution is highly biased, being found only the skin. A study by Aono et al., in which the canonical TCR junctions of the Vγ5/Vδ1 subset were disrupted by aberrant expression of the TdT gene, showed that only the now-rare Vγ5/Vδ1 cells having canonical TCR junctions took up permanent residence in the skin of these mice, though all appeared transiently, which could indicate that perhaps the Vγ5/Vδ1 TCR acts as a “homing” receptor to retain these cells in the epidermis (Aono et al., Immunol 99:489-497 (2000)). Third, a number of studies have suggested that γδ T cells are in general more easily and quickly activated than are αβ T cells (Leclercq et al., Scand J Immunol 36:833-841 (1992); Skeen et al., J Exp Med 178:985-996 (1993); Lahn et al., J Immunol 160:5221-5230 (1998); Tough et al., J Exp Med 187:357-365 (1998)), which could indicate that some of their responses are made independently of the TCR, or after TCR-independent activation events take place.

Moreover, other than the TCR, a number of receptors have been identified on γδ T cells or γδ T cell subsets that could operate early in immune or inflammatory responses. For example, a large subset of bovine γδ T cells express a member of the cysteine-rich scavenger receptor family known as WC1 (Hanby-Flarida et al., Immunology 88:116-123 (1996)). Scavenger receptors on macrophages bind and internalize modified lipoproteins, and are thought to be capable of activating macrophages (Haworth et al., J Exp Med 186:1431-1439 (1997)). A second example concerns the mouse Vγ6/Vδ1 subset that is the focus of this study; during infection, these cells, but not other γδ T cells, express Toll-like receptor 2 (TLR2) mRNA (Mokuno et al., J Immunol 165:931-940 (2000)). This receptor detects certain bacterial products and acts as an activating receptor for macrophages. A third example is NKG2D, a receptor found on most members of the human Vδ1⁺ subset. Expressed by both NK cells and T cells, this molecule transduces an activation signal when it binds to its ligands, in particular the stress-induced MHC class Ib molecule MICA (Bauer et al., Science 285:727-729 (1999)). Co-engagement NKG2D along with the TCR is necessary to activate cytolytic activity in the case of CD8⁺ αβ T cells (Groh et al., Nature Immunol 2:255-260 (2001)), whereas for NK cells, NKG2D engagement alone may be sufficient. Mouse epidermal Vγ5/Vδ1 γδ T cells were likewise recently shown to express NKG2D, and to lyse tumor cells expressing the MICA analogue Rae-1 (Girardi et al., Science 294:605-609 (2001)), although the lysis was less efficient when the TCR was blocked.

Despite these numerous arguments against the need for the TCR in γδ T cells activation, the experiments described herein indicate that the TCR is in fact essential for at least two aspects of the response of the mouse Vγ6/Vδ1 subset. Injecting a soluble version of this TCR into mice in an attempt to out compete the normal TCR for binding to its hypothetical ligand during Listeria infection, the present inventors found that both expansion and functional activation of the Vγ6/Vδ1⁺ γδ T cell subset no longer take place. These findings indicate that γδ TCR stimulation acts as positive rather than a negative signal, as has been suggested for γδ T cell activation. Finally, this experimental approach allowed the present inventors for the first time to directly examine the functional role of the Vγ6/Vδ1 γδ T cell subset, without also affecting the responses of other γδ cells. The results described herein indicate that this subset has an anti-inflammatory effect in this disease, since the ability of the mice to clear Listeria was substantially enhanced in mice in which Vγ6/Vδ1 responses were blocked by treatment with soluble Vγ6/Vδ1 TCR, as compared to untreated or sham-treated mice. This confirms other reports in a number of different disease models in which an anti-inflammatory effect was also postulated for this subset (Ando et al., J Immunol 167:3740-3045 (2001); Ikebe et al., Immunol 102:94-102 (2001); Mukasa et al., J Immunol 159:5787-5794 (1997); Roark et al., J Immunol 156:2214-2220 (1996)), although some studies suggested a pro-inflammatory effect instead (Mokuno et al., J Immunol 165:931-940 (2000); Takano et al., J Immunol 161:3019-3025 (1998)).

The present inventors' success in inhibiting γδ T cell responses by injection of soluble TcRs was a surprise, given the often demonstrated weak affinity of the αβ TCR for MHC/peptide ligands. In fact, the previous experiences of others suggested that such low affinity precludes functional blockage on any practical level even when using multimerized soluble αβ TcRs. Because of their structural similarities, the γδ T cell receptor was expected to have a similarly low ligand affinity, low enough that it would not be feasible to use soluble T cell receptor as a competitive blocker to prevent activation of a γδ T cell subset.

Therefore, the present inventors' results suggest that the γδ TCR/ligand affinity is substantially higher, at least for the Vγ6/Vδ1 γδ TCR. Affinities of TCR/ligand interactions involving polyclonally expressed TcRs have not been examined, but a study by Crowley et al., in which the affinity of a clonal γδ TCR for the class Ib MHC molecule T10 was directly measured, supports the idea in that the γδ TCR/T10 affinity was found to exceed the average αβ TCR/ligand affinity by ˜100 fold (i.e. a dissociation constant of ˜10⁻⁷M vs. ˜10⁻⁵M for a typical αβ TCR) (Crowley et al., J Exp Med 185:1223-1230 (1997); Alam et al., Nature 381:616-620 (1996); Matsui et al., Proc Natl Acad Sci USA 91:12862-12866 (1994); Seibel et al., J Exp Medicine 185:1919-1927 (1997)).

The present inventors also asked whether the effects reported herein using soluble Vγ6/Vδ1 TCR might instead actually be caused by something other than blocking of the ligand. Non-specific inhibition seems improbable, since neither an αβ TCR or even a very closely related γδ TCR (Vγ5/Vδ1) had any effect in the Vγ6/Vδ1 regulated system. Moreover, other γδ T cell subsets expanded normally in mice treated with the soluble Vγ6/Vδ1 TCR. These other subsets provided an internal control for any potential non-specific inhibitory effects peculiar to the soluble Vγ6/Vδ1 TCR. Another possibility might be that treating the mice with soluble Vγ6/Vδ1 TCR induced production of anti-Vγ6/Vδ1 antibody in the mice. This is unlikely for two reasons. First, the Vγ6/Vδ1 TCR is a “self” molecule normally present in these mice, and they should therefore be tolerant to soluble Vγ6/Vδ1 TCR. Even if the soluble form of this receptor contained some immunogenic portions, e.g., as a result of the introduced BirA site or foreign glycosylation derived from the insect cells used to grow the TCR preparations, antibodies produced against them would not be expected to affect the normal cell-bound Vγ6/Vδ1 TCR, which lacks these modifications. Second, the time period between introduction of the soluble TCR and determination of the effect was too short to allow antibody development to progress to any measurable degree—as short as 3 days, in the experiments in which bacterial clearance was measured. Therefore, it can be concluded that ligand interference is the explanation for the observed effects of the soluble Vγ6/Vδ1 TCR.

The use of soluble TCR described herein has some inherent advantages over other experimental methods of blocking or eliminating γδ T cell subsets. First, antibody against the TCR of interest need not be available, and indeed, lack of a specific mAb was one of the main reasons the inventors first chose to examine the enigmatic mouse Vγ6/Vδ1 subset. Second, depletion of γδ T cell subsets by mAb injection precludes that transient activation of the cells of interest will also occur. When using a soluble γδ TCR to block activation of the cells, the γδ T cells of interest are in contrast never even touched, since the soluble TCR should bind only to the ligand, presumably expressed by other cells. It also leaves their TcRs intact and available for identifying the cells. The use of gene inactivation to destroy certain γδ T cell subsets has other potential problems, in that it generates an animal in which development may have been altered by congenital lack of this cell type, a real concern with γδ T cells, for which organ homeostasis effects have already been reported (Findly et al., Eur J Immunol 23:2557-2564 (1993); King et al., J Immunol 162:5033-5036 (1999); Lahn et al., Nature Medicine 5:1150-1156 (1999)). Third, soluble γδ TcRs may enable for the first time the identification of the natural ligands for γδ TcRs, in particular those that appear to be inducible host molecules that drive the responses of entire subsets (reviewed in O'Brien et al., J Immunol 165:6472-6479 (2000)), which have until now defied identification. Additionally, the specific nature of the cellular responses that are blocked indicates that soluble γδ TcRs may be beneficial for use as drugs, as the understanding of how these cells carry out their functions grows. For instance, although it is not yet known how the anti-inflammatory effect of the Vγ6/Vδ1 subset is mediated, it appears to play a negative role in bacterial clearance during Listeria infection, but the inventors, without being bound by theory, believe that it plays a positive role in reducing or preventing tissue damage in autoimmune inflammatory responses. Manipulation of γδ T cell subset responses may thus be a neutral way to alter an immune or inflammatory response, by modulating the body's own regulatory controls.

The method of the present invention includes the administration of a soluble γδ T cell receptor (TCR) to a mammal to regulate a γδ T cell ligand-mediated immune response in the mammal. By binding to the ligand, the soluble γδ T cell receptor serves as a competitive inhibitor of the endogenous γδ T cells bearing the same receptor, which, as discussed above, the inventors have surprisingly shown can regulate the γδ T cell immune response in the mammal.

A “γδ T cell” is a distinct lineage of T lymphocytes found in mammalian species and birds that expresses a particular antigen receptor (i.e., T cell receptor or TCR) that includes a γ chain and a δ chain. The γ and δ chains are distinguished from the α and β chains that make up the TCR of the perhaps more commonly referenced T cells known as “αβ T cells”. The γδ heterodimer of the γδ T cells is expressed on the surface of the T cell and, like the αβ heterodimer of αβ T cells, is associated with the CD3 complex on the cell surface. The γ and δ chains of the γδ T cell receptor should not be confused with the γ and δ chains of the CD3 complex. According to the present invention, the terms “T lymphocyte” and “T cell” can be used interchangeably herein.

According to the present invention, a “soluble” T cell receptor is a T cell receptor consisting of the chains of a full-length (e.g., membrane bound) receptor, except that, minimally, the transmembrane region of the receptor chains are deleted or mutated so that the receptor, when expressed by a cell, will not associate with the membrane. Most typically, a soluble receptor will consist of only the extracellular domains of the chains of the wild-type receptor (i.e., lacks the transmembrane and cytoplasmic domains).

γδ T cell receptors are composed of a heterodimer of a γ chain and a δ chain. At the time of the invention, multiple different functional murine γ chains, murine δ chains, human γ chains, and human δ chains were known. Various specific combinations of γ and δ chains are preferred for use in the soluble γδ T cell receptors of the invention, and particularly those corresponding to γδ T cell subsets that are known to exist in vivo, but it is to be understood that soluble γδ T cell receptors having virtually any combination of γ and δ chains are also contemplated for use in the present invention. Preferably, soluble γδ T cell receptors comprise γ and δ chains derived from the same animal species (e.g., murine, human).

A soluble γδ T cell receptor useful in the invention typically is a heterodimer comprising a γ chain and a δ chain, but multimers (e.g., tetramers) comprising two different γδ heterodimers or two of the same γδ heterodimers are also contemplated for use in the present invention. As set forth above, preferably, γ and δ chains from the same species of mammal (e.g., murine, human) are combined to form a γδ heterodimer. Suitable murine γ chains for use in the present invention include, but are not limited to:

Vγ1 (SEQ ID NO:11 (cDNA); SEQ ID NO:12 (amino acid)) (WHO Designation mGV5S1; GenBank Accession No. M12832);

Vγ4 (SEQ ID NO:13 (cDNA); SEQ ID NO:14 (amino acid)) (WHO Designation mGV3; GenBank Accession No. M13336);

Vγ5 (SEQ ID NO:15 (cDNA); SEQ ID NO:16 (amino acid)) (WHO Designation mGV1S1; GenBank Accession No. M13337);

Vγ6 (SEQ ID NO:17 (cDNA); SEQ ID NO:18 (amino acid)) (WHO Designation mGV2; GenBank Accession No. M13338);

Vγ7 (SEQ ID NO:19 (cDNA); SEQ ID NO:20 (amino acid)) (WHO Designation mGV4; GenBank Accession No. M71214 or Z48594).

Suitable murine δ chains for use in the present invention include, but are not limited to:

Vδ1 (SEQ ID NO:21 (cDNA); SEQ ID NO:22 (amino acid)) (WHO Designation mDV101; GenBank Accession No. M23545);

Vδ5 (SEQ ID NO:23 (cDNA); SEQ ID NO:24 (amino acid)) (WHO Designation mDV105; GenBank Accession No. M37282);

Vδ6.3 (SEQ ID NO:25 (cDNA); SEQ ID NO:26 (amino acid)) (WHO Designation mADV7S1; GenBank Accession No. X02935).

Suitable human γ chains for use in the present invention include, but are not limited to:

Vγ8 (SEQ ID NO:27 (cDNA); SEQ ID NO:28 (amino acid)) (WHO Designation hGV1; GenBank Accession No. M13434; note this receptor chain has also been referred to as Vγ1 in humans);

Vγ9 (SEQ ID NO:29 (cDNA); SEQ ID NO:30 (amino acid)) (WHO Designation hGV2; GenBank Accession No. X72500; note this receptor chain has also been referred to as Vγ2 in humans).

Suitable human δ chains for use in the present invention include, but are not limited to:

Vδ2 (SEQ ID NO:31 (cDNA); SEQ ID NO:32 (amino acid)) (WHO Designation hDV102; GenBank Accession No. X72501);

Vδ3 (SEQ ID NO:33 (cDNA); SEQ ID NO:34 (amino acid)) (WHO Designation hDV103; GenBank Accession No. X13954);

Vδ4 (SEQ ID NO:35 (cDNA); SEQ ID NO:36 (amino acid)) (WHO Designation hADV6; GenBank Accession No. M21624).

The content of each of the GenBank Accession Nos. set forth above is incorporated herein by reference in its entirety; World Health Organization (WHO) Designations are also given for clarity. A more complete list of mouse and human Vγ and Vδ chains, including WHO designations sequence Accession Nos., is described in Arden et al., 1995, Immunogenetics 42:501-530; and Arden et al., 1995, Immunogenetics 42:455-500; each of which is incorporated herein by reference in its entirety.

Preferred combinations of murine γ and δ chains include, but are not limited to, Vγ6/Vδ1, Vγ5/Vδ1, Vγ1/Vδ6.3, Vγ1/Vδ6B, Vγ1/Vδ4, Vγ1/Vδ5, Vγ4/Vδ4, Vγ4/Vδ5, Vγ7/Vδ5, Vγ7/Vδ4, Vγ7/Vδ6.3, Vγ7/Vδ6B. Preferred combinations of human γ and δ chains include, but are not limited to, Vγ9/Vδ2, Vγ9/Vδ1, Vγ9/Vδx and Vγ8/Vδx, where Vδx is any human Vδ chain.

Certain subsets of murine γδ T cell receptors have equivalents or some biological relation to certain subsets of human γδ T cell receptors. For example, murine Vγ1 is approximately equivalent to human Vγ9; murine Vγ4 has no human equivalent, but is more related to human Vγ9 than to human Vγ8; murine Vγ5 has no human equivalent, and is about equally related to human Vγ8 and human Vγ9; murine Vγ6 has no human equivalent and is about equally related to human Vγ8 and human Vγ9; murine Vγ7 is approximately equivalent to human Vγ8. Murine Vδ1 is approximately equivalent to human Vδ2; mouse Vδ5 is most nearly related to human Vδ3; murine Vδ6.3 is most nearly related to human Vδ4.

It is also noted that human Vδ1⁺ T cells (usually combined with human Vγ9) have been found as intraepithelial lymphocytes in various tissues. This is also true of Vγ4⁺ murine γδ T cells, to some extent. Therefore, without being bound by theory, murine Vγ4⁺ γδ T cells may share some biological functions with human Vδ1⁺ (e.g., Vγ9/Vδ1) γδ T cells.

γδ T cell receptors may be selected for use in the invention based on their location and function relative to the disease or condition to be treated. Depending on the condition is or disease (or location in the body), enhancement or inhibition of a given γδ T cell subset may be desired. For example, the Vγ6/Vδ1 subset has been reported to predominate among the T cells normally present in certain epithelial sites, in particular the uterus (Itohara et al., Nature 343:754-757 (1990)) and the lung (Hayes et al., J. Immunol. 156:2723-2729 (1996)). A preferential expansion of Vγ6/Vδ1⁺ cells has also been noted in a variety of experimental systems which induce an inflammatory response. These include Listeria infection of the liver (Roark et al., J. Immunol. 156:2214-2220 (1996)) and kidney (Ikebe et al., Immunol. 102:94-102 (2001)), autoimmune and infection-induced orchitis (Mukasa et al., J. Immunol. 159:5787-5794 (1997); Mukasa et al., J. Immunol. 162:4910-4913 (1999)), experimental allergic encephalomyelitis (EAE) (Olive, C., Immunol. Cell Biol. 75:102-106 (1997)), drug-induced kidney damage (Ando et al., J. Immunol. 167:3740-3045 (2001)), and E. coli intraperitoneal infection (Matsuzaki et al., Eur. J. Immunol. 29:3877-3886 (1999)). The subset also expands in the uterus during pregnancy. Indeed, both γδ T cells expressing T cell receptors with a murine Vγ1 chain and γδ T cell expressing T cell receptors with a murine Vγ6 chain have been shown to be expanded during inflammation, including Listeria infection. Without being bound by theory, the present inventors believe that at least the Vγ6-expressing subset actually reduces or prevents tissue damage in autoimmune and inflammatory responses, but that this effect actually impedes the clearance of bacteria during a bacterial infection (i.e., the anti-inflammatory regulation by this subset inhibits the proinflammatory modulators that would otherwise work to clear the infectious agent). Therefore, blocking this effect by using a soluble γδ T cell receptor according to the present invention (e.g., soluble receptors comprising Vγ6 or Vγ1) allows proinflammatory responses to clear the infectious bacteria. In other conditions, such as autoimmune disease, where a proinflammatory response can be deleterious, the invention may include the inhibition of a different subset of γδ T cells so that the activity of subsets such as the Vγ1⁺ or Vγ6⁺ subsets is effectively augmented, e.g., by inhibition of competing, proinflammatory γδ T cell subsets. The mouse Vγ1⁺ subset appears to protect against inflammatory damage in a myocarditis model, whereas the mouse Vγ4⁺ subset appears to promote inflammatory damage in the same model (Huber et al., J. Immunol. 165:4174-4181 (2000)). Therefore, in one embodiment, the method of the invention includes providing a soluble γδ T cell receptor comprising a Vγ4 chain, in order to inhibit the proinflammatory damage caused by endogenous Vγ4+ γδ T cells, and with the potential added benefit of augmenting the activity of the protective Vγ1+ γδ T cells.

The invention includes application of any of these guidelines to the equivalent or functionally related human γδ T cell subsets. For example, it has been reported that in humans, Vγ9/Vδ2 γδ T cells tend to be cytolytic and produce Th1-type cytokines (i.e., these T cells tend to have a proinflammatory phenotype) (Fisch et al., Eur. J. Immunol. 27:3368-3379). Therefore, inhibition of this human subset (e.g., by administration of a soluble γδ T cell receptor comprising a Vγ9 chain and/or a Vδ2 chain) in conditions or tissues where inhibition of the proinflammatory activity is desired is an embodiment of the invention. Similarly, in conditions where a proinflammatory response is desired, one may augment the activity of this subset by administering a soluble γδ T cell receptor for a non-Vγ9+, non-Vδ2+ γδ T cell that resides in the same tissue or is expanded in the same condition, so that there is less competition or inhibition of the activity of the Vγ9/Vδ2 γδ T cells.

Another subset of Vγ⁺ T cells in the murine lung express the CD8 αβ heterodimer. Without being bound by theory, the present inventors believe that γδ T cells expressing a CD8 αβ heterodimer, and particularly γδ T cells expressing Vγ4 and a CD8 αβ heterodimer (or in humans, γδ T cells expressing Vδ1—see discussion above), may be at least one primary regulatory γδ T cell subset that contributes to the reduction of airway hyperresponsiveness (AHR) in vivo. Therefore, enhancement or augmentation of this subset, for example by reducing the activity of another γδ T cell subset that competes with the Vγ4 subset or that is simply found in the same tissue, is one embodiment of the invention. Given these examples, other uses of various soluble γδ T cell receptors to treat different conditions and diseases will be apparent to those of skill in the art.

Based on the discussion herein, it will now be apparent to those of skill in the that the method of the present invention can be designed to inhibit and/or attempt to augment the activity of any selected γδ T cell subset (or multiple subsets) in order to achieve the desired effect in a given tissue and condition. One simply produces and administers a soluble γδ T is cell receptor to block the activity of the endogenous γδ T cells having the same receptor or at least one of the same receptor chains (Vγ or Vδ).

Soluble γδ T cell receptors of the present invention can be produced by any suitable method known to those of skill in the art, and are most typically produced recombinantly. According to the present invention, a recombinant nucleic acid molecule useful for producing a soluble γδ T cell receptor typically comprises a recombinant vector and a nucleic acid sequence encoding one or more segments (e.g., chains) of a γδ T cell receptor as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences which encode a protein of interest (e.g., the T cell receptor chains) or which are useful for expression of the nucleic acid molecules. The vector can be either RNA or DNA, either prokaryotic or eukaryotic, and typically is a plasmid.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

One or more recombinant molecules of the present invention can be used to produce an encoded product (e.g., a soluble γδ T cell receptor) of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cell that can be transfected. Host cells can be either untransfected cells or cells that are already transfected with at least one other recombinant nucleic acid molecule. Resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins produced according to the present invention are preferably retrieved in “substantially pure” form. As used herein, “substantially pure” refers to a purity that allows for the effective use of the soluble γδ T cell receptor in a composition and method of the present invention.

By way of example, recombinant constructs containing the relevant γ and δ genes (e.g., nucleic acid sequences encoding the desired portions of the γ and δ chains of a γδ T cell receptor) can be produced by PCR of T cell receptor cDNAs derived from a source of γδ T cells (e.g., hybridomas, clones, transgenic cells) that express the desired receptor. The PCR amplification of the desired γ and δ genes can be designed so that the transmembrane and cytoplasmic domains of the chains will be omitted (i.e., creating a soluble receptor). Preferably, portions of the genes that form the interchain disulfide bond are retained, so that the γδ heterodimer formation is preserved. In addition, if desired, sequence encoding a selectable marker for purification or labeling of the product or the constructs can be added to the constructs. Amplified γ and δ cDNA pairs are then cloned, sequence-verified, and transferred into a suitable vector, such as a baculoviral vector containing dual baculovirus promoters (e.g., pAcUW51, Pharmingen Corp., San Diego, Calif.).

The soluble γδ TCR DNA constructs are then co-transfected into a suitable host cell (e.g., in the case of a baculoviral vector, into suitable insect host cells) which will express and secrete the recombinant receptors into the supernatant, for example. Culture supernatants containing soluble γδ TCRs can then be purified using various affinity columns, such as anti-Cδ (GL3) sepharose affinity columns. The products can be concentrated and stored. A detailed description of an exemplary procedure for the production of soluble γδ T cell receptors is provided in the Examples section. It will be clear to those of skill in the art that other methods and protocols can be used to produce soluble T cell receptors for use in the present invention, and such methods are expressly contemplated for use herein.

A soluble γδ T cell receptor of the invention is typically administered to a mammal as a composition which includes a pharmaceutically acceptable carrier. Pharmaceutically acceptable carriers include pharmaceutically acceptable excipients and/or delivery vehicles for administering a given agent (i.e., the soluble receptor) to a patient. As used herein, a pharmaceutically acceptable carrier refers to any substance suitable for delivering a soluble receptor useful in the method of the present invention to a suitable in vivo or ex vivo site. Preferred pharmaceutically acceptable carriers are capable of maintaining the soluble receptor and any other agents included in the composition in a form that, upon arrival of the soluble receptor in the patient and/or at a target cell (if the procedure is ex vivo), the agent is capable of interacting with its target (i.e., a ligand for the γδ T cell) such that the activity of the endogenous γδ T cell is reduced or prevented, or so that the activity of the ligand is reduced or inhibited. Suitable excipients of the present invention include excipients or formularies that transport or help transport, but do not specifically target an agent to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters, glycols and dry-powder inhalers. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into an animal. As used herein, a controlled release formulation comprises a soluble T cell receptor and any other agents included in a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. In one embodiment, when the route of delivery is inhaled, a composition or agent of the present invention can be delivered by an inhaler device.

A pharmaceutically acceptable carrier which is capable of targeting can be referred to as a “delivery vehicle.” Delivery vehicles of the present invention are capable of delivering a composition including a soluble γδ T cell receptor to a target site in a mammal. A “target site” refers to a site in a mammal to which one desires to deliver a therapeutic composition. For example, a target site can be any cell which is targeted by direct injection or delivery using antibodies (e.g., monospecific, chimeric or bispecific antibodies) or liposomes, for example. A delivery vehicle of the present invention can be modified to target to a particular site in a mammal (e.g., a particular tissue type), thereby targeting and making use of a soluble γδ T cell receptor at that site. Suitable modifications include manipulating the chemical formula of the lipid portion of the delivery vehicle and/or complexing the receptor with an agent that is capable of specifically targeting the receptor to a preferred site, for example, a preferred cell or tissue type. Targeting refers to causing a soluble receptor of the invention to contact or come into close proximity with a particular cell by the interaction of the targeting agent with a molecule on the surface of the cell. Suitable targeting compounds include ligands capable of selectively (i.e., specifically) binding another molecule at a particular site (e.g., antibodies, antigens, receptors and receptor ligands, glycoproteins).

One embodiment of the present invention relates to a composition for regulating a γδ T cell-mediated immune response in a mammal, comprising: (a) a soluble γδ T cell receptor as previously described herein; and (b) an agent that regulates inflammation in said mammal. According to the invention, the agent of (b) can include any agent that is useful for treating a given disease or condition the mammal has or is at risk of developing. Such agents include, but are not limited to, pharmaceuticals specific for the condition, cytokine antagonists (e.g., anti-cytokine antibodies, soluble cytokine receptors), cytokine receptor antagonists (e.g., anti-cytokine receptor antibodies), cytokines, anticholinergics, immunomodulating drugs, leukotriene synthesis inhibitors, leukotriene receptor antagonists, glucocorticosteroids, steroid chemical derivatives, anti-cyclooxygenase agents, anti-cholinergic agents, beta-adrenergic agonists, methylxanthines, anti-histamines, cromones, zyleuton, surfactants, anti-thromboxane reagents, anti-serotonin reagents, ketotiphen, cytoxin, cyclosporin, methotrexate, macrolide antibiotics, heparin, low molecular weight heparin, and mixtures thereof.

In accordance with the present invention, acceptable protocols to administer a soluble γδ T cell receptor, including the route of administration and the effective amount of the soluble receptor to be administered to an animal, can be determined and accomplished by those skilled in the art. An agent of the present invention can be administered in vivo or ex vivo. Suitable in vivo routes of administration can include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. Some particularly preferred routes of administration include, intravenous, intraperitoneal, subcutaneous, intradermal, intranodal, intramuscular, transdermal, inhaled, intranasal, rectal, vaginal, urethral, topical, oral, intraocular, intraarticular, intracranial, and intraspinal. Combinations of routes of delivery can be used and in some instances, may enhance the therapeutic effects of the composition. The best mode of administration will depend on the disease or condition to be treated and particularly, the location in the patient of the tissue(s) affected by the disease or condition.

Ex vivo refers to performing part of the administration step outside of the patient, such as by removing cells from a patient, culturing such cells in vitro with a soluble γδ T cell receptor, and returning the cells, or a subset thereof to the patient.

A suitable single dose of a soluble γδ T cell receptor to administer to a mammal is a dose that is capable of reducing or inhibiting the activity of the endogenous γδ T cells having the same γδ T cell receptor when the soluble receptor is administered one or more times over a suitable time period. A preferred single dose of a soluble receptor typically comprises between about 0.01 microgram×kilogram⁻¹ and about 10 milligram×kilogram⁻¹ body weight of an animal. A more preferred single dose of soluble receptor comprises between about 1 microgram×kilogram⁻¹ and about 10 milligram×kilogram⁻¹ body weight of an animal. An even more preferred single dose of a soluble receptor comprises between about 5 microgram×kilogram⁻¹ and about 7 milligram×kilogram⁻¹ body weight of an animal. An even more preferred single dose of a soluble receptor comprises between about 10 microgram×kilogram⁻¹ and about 5 milligram×kilogram⁻¹ body weight of an animal. A particularly preferred single dose of a soluble receptor comprises between about 0.1 milligram×kilogram⁻¹ and about 5 milligram×kilogram⁻¹ body weight of an animal, if the soluble receptor is delivered by aerosol. Another particularly preferred single dose of a soluble receptor comprises between about 0.1 microgram×kilogram⁻¹ and about 10 microgram×kilogram⁻¹ body weight of an animal, if the soluble receptor is delivered parenterally.

In general, the biological activity or biological action of a γδ T cell receptor or of a γδ T cell expressing such a receptor refers to any function(s) exhibited or performed by the receptor or cell that is ascribed to the naturally occurring receptor or cell as measured or observed in vivo (i.e., in the natural physiological environment of the protein) or in vitro (i.e., under laboratory conditions). Administration of a soluble γδ T cell receptor according to the present invention may have a variety of results on the activity of the endogenous γδ T cells expressing the same receptor, including, but not limited to, inhibition of binding of the endogenous receptor to its ligand, inhibition of expansion of a subset of γδ T cells that have that receptor, inhibition of biological activities in the mammal that are associated with the binding of the endogenous receptor to its ligand (e.g., cytokine production, production of other inflammatory or anti-inflammatory modulators, T cell proliferation and expansion, recruitment of other cells to the local environment, upregulation or downregulation of cell surface molecules, induction of apoptosis in target cells). Changes which result in a decrease in the expression or activity of the receptor or cell, can be referred to as inactivation (complete or partial), downregulation, inhibition, reduction, or decreased activity. Similarly, changes which result in an increase in the expression or activity of the receptor or cell can be referred to as amplification, augmentation, overproduction, activation, enhancement, upregulation or increased activity.

Changes in the expression or activity of γδ T cell receptors and T cells expressing such receptors can be measured using any technique known to those of skill in the art for evaluating the presence and expression of a cell surface molecule, and/or the activity of a T lymphocyte and particularly, a γδ T lymphocyte. Such techniques include, but are not limited to, detection of expression of specific receptors using protein or nucleic acid detection methods, measurement of changes in the numbers of cells, measurement of changes in T lymphocyte biological function. For example, characteristics of T cell receptor expression and T cell activation can be determined by a method including, but not limited to: measuring receptor expression (e.g., by flow cytometry, immunoassay, RNA assays); measuring cytokine production by the T cell (e.g., by immunoassay or biological assay); measuring intracellular and/or extracellular calcium mobilization (e.g., by calcium mobilization assays); measuring T cell proliferation (e.g., by proliferation assays such as radioisotope incorporation); measuring upregulation of cytokine receptors on the T cell surface, including IL-2R (e.g., by flow cytometry, immunofluorescence assays, immunoblots, RNA assays); measuring upregulation of other receptors associated with T cell activation on the T cell surface (e.g., by flow cytometry, immunofluorescence assays, immunoblots, RNA assays); measuring reorganization of the cytoskeleton (e.g., by immunofluorescence assays, immunoprecipitation, immunoblots); measuring upregulation of expression and activity of signal transduction proteins associated with T cell activation (e.g., by kinase assays, phosphorylation assays, immunoblots, RNA assays); and, measuring specific effector functions of the T cell (e.g., by proliferation assays). Methods for performing each of these measurements are well known to those of ordinary skill in the art, many are described in detail or by reference to publications herein, and all such methods are encompassed by the present invention.

According to the present invention, the therapeutic method of the present invention is primarily directed to the regulation of a γδ T cell-mediated immune response in a mammal with the presumed, but not absolutely required, goal of providing some therapeutic benefit to the mammal. Modulating the γδ T cell-mediated immune response in a mammal in the absence of obtaining some therapeutic benefit is useful for the purposes of identifying γδ T cell ligands, for example, the identification of which to date has been somewhat elusive, for determining factors involved (or not involved) in a given disease, and/or preparing a patient to more beneficially receive another therapeutic composition that may provide a therapeutic benefit. In a preferred embodiment, however, the method of the present invention is directed to the regulation of a γδ T cell-mediated immune response in order to provide some therapeutic benefit to a patient. As such, a therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a therapeutic composition of the present invention, when administered to a patient, to prevent a disease from occurring and/or to cure or to treat the disease by alleviating disease symptoms, signs or causes. As such, to protect a patient from a disease includes both preventing disease occurrence (prophylactic treatment) and treating a patient that has a disease or that is experiencing initial symptoms or later stage symptoms of a disease (therapeutic treatment). In particular, protecting a patient from a disease or enhancing another therapy is accomplished by regulating a γδ T cell-mediated or γδ T cell ligand-mediated immune response in the patient such that a beneficial effect is obtained. A s beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

Conditions to treat using methods of the present invention include any condition or disease in which it is or may be useful to regulate γδ T cell activity. Such conditions include, but are not limited to, any condition in which γδ T cells can be regulated and preferably, includes diseases characterized by expansion or inhibition of one or more specific subsets of γδ T cells. Such conditions include, but are not limited to: intestinal conditions is (e.g., Crohn's disease, ischemic colitis, irritable bowel disease, colon cancer); inflammatory lung conditions (e.g., airway hyperresponsiveness, pneumonia, tuberculosis, primary or metastatic lung tumors); inflammatory skin conditions (e.g., skin lesions caused by bacterial or viral infection, laceration, skin cancer); inflammation of the reproductive tract (e.g., bacterial or viral infections that involve the epithelial mucosal lining, tubal infections, preventing tubal factor infertility, ovarian cancer, cervical cancer, uterine cancer, prostate cancer, testicular cancer); myocarditis, or Listeria infection.

When the condition is myocarditis, the soluble γδ T cell receptor used is preferably a Vγ4+ T cell receptor, so that endogenous γδ T cells expressing such receptor are inhibited from binding to their ligand. This preferably results in an increase in the activity of a γδ T cell subset that expresses Vγ1, which is believed to have a therapeutic benefit in this condition.

When the condition is general inflammation or alternatively, a condition in which inhibition of inflammatory processes may be deleterious for pathogen clearance, such as infection with Listeria, the soluble γδ T cell receptor is preferably a Vγ1⁺ or a Vγ6⁺ T cell receptor, so that endogenous γδ T cells expressing such a receptor are inhibited from binding to their ligand.

When the condition is airway hyperresponsiveness (AHR) associated with inflammation, the soluble γδ T cell receptor is preferably not a Vγ4⁺ T cell receptor, so that endogenous γδ T cells bearing such a receptor are allowed to act and inhibit AHR. Such a soluble receptor can include Vγ1⁺ or Vγ6⁺ T cell receptors, for example. With regard to AHR, this condition refers to any measurable reduction in airway hyperresponsiveness and/or any reduction of the occurrence or frequency with which airway hyperresponsiveness occurs in a patient. A reduction in AHR can be measured using any of the above-described techniques or any other suitable method known in the art. Preferably, airway hyperresponsiveness, or the potential therefore, is reduced, optimally, to an extent that the animal no longer suffers discomfort and/or altered function resulting from or associated with airway hyperresponsiveness. To prevent airway hyperresponsiveness refers to preventing or stopping the induction of airway hyperresponsiveness before biological characteristics of airway hyperresponsiveness as discussed above can be substantially detected or measured in a patient.

AHR can be measured by a stress test that comprises measuring an animal's respiratory system function in response to a provoking agent (i.e., stimulus). AHR can be measured as a change in respiratory function from baseline plotted against the dose of a provoking agent (a procedure for such measurement and a mammal model useful therefore are described in detail below in the Examples). Respiratory function can be measured by, for example, spirometry, plethysmograph, peak flows, symptom scores, physical signs (i.e., respiratory rate), wheezing, exercise tolerance, use of rescue medication (i.e., bronchodialators) and blood gases. In humans, spirometry can be used to gauge the change in respiratory function in conjunction with a provoking agent, such as methacholine or histamine. In humans, spirometry is performed by asking a person to take a deep breath and blow, as long, as hard and as fast as possible into a gauge that measures airflow and volume. The volume of air expired in the first second is known as forced expiratory volume (FEV₁) and the total amount of air expired is known as the forced vital capacity (FVC). In humans, normal predicted FEV₁ and FVC are available and standardized according to weight, height, sex and race. An individual free of disease has an FEV₁ and a FVC of at least about 80% of normal predicted values for a particular person and a ratio of FEV₁/FVC of at least about 80%. Values are determined before (i.e, representing a mammal's resting state) and after (i.e., representing a mammal's higher lung resistance state) inhalation of the provoking agent. The position of the resulting curve indicates the sensitivity of the airways to the provoking agent.

The effect of increasing doses or concentrations of the provoking agent on lung function is determined by measuring the forced expired volume in 1 second (FEV₁) and FEV₁ over forced vital capacity (FEV₁/FVC ratio) of the mammal challenged with the provoking agent. In humans, the dose or concentration of a provoking agent (i.e., methacholine or histamine) that causes a 20% fall in FEV₁ (PD₂₀FEV₁) is indicative of the degree of AHR. FEV₁ and FVC values can be measured using methods known to those of skill in the art.

Pulmonary function measurements of airway resistance (R_{L}) and dynamic compliance (C_{dyn} or C_{L}) and hyperresponsiveness can be determined by measuring transpulmonary pressure as the pressure difference between the airway opening and the body plethysmograph. Volume is the calibrated pressure change in the body plethysmograph and flow is the digital differentiation of the volume signal. Resistance (R_{L}) and compliance (C_{L}) are obtained using methods known to those of skill in the art (e.g., such as by using a recursive least squares solution of the equation of motion). The measurement of lung resistance (R_{L}) and dynamic compliance (C_{L}) are described in detail in the Examples. It should be noted that measuring the airway resistance (R_{L}) value in a non-human mammal (e.g., a mouse) can be used to diagnose airflow obstruction similar to measuring the FEV₁ and/or FEV₁/FVC ratio in a human.

A variety of provoking agents are useful for measuring AHR values. Suitable provoking agents include direct and indirect stimuli. Preferred provoking agents include, for example, an allergen, methacholine, a histamine, a leukotriene, saline, hyperventilation, exercise, sulfur dioxide, adenosine, propranolol, cold air, an antigen, bradykinin, acetylcholine, a prostaglandin, ozone, environmental air pollutants and mixtures thereof. Preferably, Mch is used as a provoking agent. Preferred concentrations of Mch to use in a concentration-response curve are between about 0.001 and about 100 milligram per milliliter (mg/ml). More preferred concentrations of Mch to use in a concentration-response curve are between about 0.01 and about 50 mg/ml. Even more preferred concentrations of Mch to use in a concentration-response curve are between about 0.02 and about 25 mg/ml. When Mch is used as a provoking agent, the degree of AHR is defined by the provocative concentration of Mch needed to cause a 20% drop of the FEV₁ of a mammal (PC_{20methacholine}FEV₁). For example, in humans and using standard protocols in the art, a normal person typically has a PC_{20methacholine}FEV₁>8 mg/ml of Mch. Thus, in humans, AHR is defined as PC_{20methacholine}FEV₁<8 mg/ml of Mch.

According to the present invention, respiratory function can also be evaluated with a variety of static tests that comprise measuring an animal's respiratory system function in the absence of a provoking agent. Examples of static tests include, for example, spirometry, plethysmographically, peak flows, symptom scores, physical signs (i.e., respiratory rate), wheezing, exercise tolerance, use of rescue medication (i.e., bronchodialators) and blood gases. Evaluating pulmonary function in static tests can be performed by measuring, for example, Total Lung Capacity (TLC), Thoracic Gas Volume (TgV), Functional Residual Capacity (FRC), Residual Volume (RV) and Specific Conductance (SGL) for lung volumes, Diffusing Capacity of the Lung for Carbon Monoxide (DLCO), arterial blood gases, including pH, P_{O2} and P_{CO2} for gas exchange. Both FEV₁ and FEV₁/FVC can be used to measure airflow limitation. If spirometry is used in humans, the FEV₁ of an individual can be compared to the FEV₁ of predicted values. Predicted FEV₁ values are available for standard normograms based on the animal's age, sex, weight, height and race. A normal animal typically has an FEV₁ at least about 80% of the predicted FEV₁ for the animal. Airflow limitation results in a FEV₁ or FVC of less than 80% of predicted values. An alternative method to measure airflow limitation is based on the ratio of FEV₁ and FVC (FEV₁/FVC). Disease free individuals are defined as having a FEV₁/FVC ratio of at least about 80%. Airflow obstruction causes the ratio of FEV₁/FVC to fall to less than 80% of predicted values. Thus, an animal having airflow limitation is defined by an FEV₁/FVC less than about 80%.

In one embodiment, the method of the present invention decreases methacholine responsiveness in the animal. Preferably, the method of the present invention results in an improvement in a mammal's PC_{20methacholine}FEV₁ value such that the PC_{20methacholine}FEV₁ value obtained before use of the present method when the mammal is provoked with a first concentration of methacholine is the same as the PC_{20methacholine}FEV₁ value obtained after use of the present method when the mammal is provoked with double the amount of the first concentration of methacholine. Preferably, the method of the present invention results in an improvement in a mammal's PC_{20methacholine}FEV₁ value such that the PC_{20methacholine}FEV₁ value obtained before the use of the present method when the animal is provoked with between about 0.01 mg/ml to about 8 mg/ml of methacholine is the same as the PC_{20methacholine}FEV₁ value obtained after the use of the present method when the animal is provoked with between about 0.02 mg/ml to about 16 mg/ml of methacholine.

In another embodiment, the method of the present invention improves an animal's FEV₁ by at least about 5%, and more preferably by between about 6% and about 100%, more preferably by between about 7% and about 100%, and even more preferably by between about 8% and about 100% of the mammal's predicted FEV₁. In another embodiment, the method of the present invention improves an animal's FEV₁ by at least about 5%, and preferably, at least about 10%, and even more preferably, at least about 25%, and even more preferably, at least about 50%, and even more preferably, at least about 75%.

In yet another embodiment, the method of the present invention results in an increase in the PC_{20methacholine}FEV₁ of an animal by about one doubling concentration towards the PC_{20methacholine}FEV₁ of a normal animal. A normal animal refers to an animal known not to suffer from or be susceptible to abnormal AHR. A patient, or test animal refers to an animal suspected of suffering from or being susceptible to abnormal AHR.

Therefore, an animal that has a disease or condition associated with inflammation, such as airway hyperresponsiveness, is an animal in which the disease or condition is measured or detected (e.g., for AHR such as by using one of the above methods for measuring airway hyperresponsiveness). To be associated with inflammation, the condition associated with inflammation described herein is apparently or obviously, directly or indirectly associated with (e.g., caused by, a symptom of, indicative of, concurrent with) an inflammatory condition or disease (i.e., a condition or disease characterized by inflammation). For AHR and diseases of the respiratory tract, typically, such an inflammatory condition or disease is at least partially characterized by inflammation of pulmonary tissues. For diseases of the reproductive tract, such an inflammatory condition or disease is at least partially characterized by inflammation of reproductive tissues, and so on. Such conditions or diseases are discussed above. An animal that is at risk of developing a particular disease or condition can be an animal that has an early symptom which is likely to be associated with at least a potential for the specified condition or disease, but does not yet display a measurable or detectable characteristic or symptom of the specified disease or condition. An animal that is at risk of developing a given disease or condition also includes an animal that is identified as being predisposed to or susceptible to such a condition or disease.

Inflammation is typically characterized by the release of inflammatory mediators (e.g., cytokines or chemokines) which recruit cells involved in inflammation to a tissue. For example, a condition or disease associated with allergic inflammation is a condition or disease in which the elicitation of one type of immune response (e.g., a Th2-type immune response) against a sensitizing agent, such as an allergen, can result in the release of inflammatory mediators that recruit cells involved in inflammation in a mammal, the presence of which can lead to tissue damage and sometimes death. Airway hyperresponsiveness associated with allergic inflammation can occur in a patient that has, or is at risk of developing, any chronic obstructive disease of the airways, including, but not limited to, asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma and parasitic lung disease. Viral-induced inflammation typically involves the elicitation of another type of immune response (e.g., a Th1-type immune response) against viral antigens, resulting in production of inflammatory mediators the recruit cells involved in inflammation in an animal, the presence of which can also lead to tissue damage.

The method of the present invention can be used in any animal, and particularly, in any animal of the Vertebrate class, Mammalia, including, without limitation, primates, rodents, livestock and domestic pets. Preferred mammals to treat using the method of the present invention include humans.

The following examples are provided for the purpose of illustration and are not intended to limit the scope of the present invention.

### EXAMPLES

Materials and Methods

Generation of Soluble γδT Cell Receptors.

Nomenclature used throughout for the murine γ chains is from that originally proposed by the Tonegawa laboratory (Maeda et al., Proc Natl Acad Sci USA 84:6539-6540 (1987)). Baculovirus constructs containing the relevant γ and δ genes were produced by PCR of TCR cDNAs derived from representative, well-characterized γδ T cell hybridomas, except for the KN6-derived Vγ4/Vδ5 TCR, for which cDNA was prepared from the spleen of a KN6 transgenic/RAG^{-/-} mouse. For each, primers were designed that would truncate the genes just before the transmembrane regions, by inserting termination codons at this point. The cysteine codon for each chain that forms the interchain disulfide bond of the TCR was preserved in each case, such that the TCR sequence ends directly after the cysteine codon for Cδ, and two codons below it for Cγ. In addition, just prior to the termination codon, the Cδ genes include a 15 codon sequence (a BSP sequence) whose product is recognized with high affinity by the E. coli enzyme Bir A which can then be used to add a biotin to the C-terminus of the soluble TCR, if desired Amplified γ and δ cDNA pairs were then cloned, sequence-verified, transferred into a vector containing dual baculovirus promoters (pAcUW51, Pharmingen Corp., San Diego, Calif.) which had been modified to include additional restriction enzyme cloning sites (pBACp10pH, gift of John Kappler, National Jewish). In each, the γ gene was cloned into the Eco RI and Bam HI sites of the polyhedrin promoter, and the δ gene into the Xho I and Bpu 1102 sites adjacent to the p10 promoter.

Oligonucleotide primers used to amplify and alter the γδ soluble TCR cDNAs were as follows (all listed 5′ to 3′):

For the Vγ6/Vδ1 TCR—Vγ6L Eco RI⁺ (GAA TTC TGC AGG ATG GGG GCT TCT; SEQ ID NO:1) with Cγ1 cys⁻ (GGA TCC TTA TTG CCA GCA AGT TGT; SEQ ID NO:2), and 5′ Vδ1-XhoI⁺ (GCC TCG AGG AAA CTA TGC TTT GGA GA; SEQ ID NO:3) with 3′ CδBSP⁻ (GCG CTC AGC TTA ACG ATG ATT CCA CAC CAT TTT CTG TGC ATC CAG AAT ATG ATG CAG GCC ATA GCA AGG CTC TGA AAT TTG; SEQ ID NO:4).

For the Vγ5/Vδ1 TCR—Vγ5L Eco RI⁺ (GG GAA TTC ACT AAA ATG TCA ACC TCT; SEQ ID NO:5) with Cγ1 cys⁻ (listed above; SEQ ID NO:2), and 5′ Vδ1-XhoI⁺ (listed above; SEQ ID NO:3) with 3′ Cδ BSP⁻ (listed above; SEQ ID NO:4).

For the Vγ1/Vδ6.3 TCR—Vγ1L-EcoRI⁺ (GGG AAT TCC TTG GGA TGC TGC TC; SEQ ID NO:6) with Cγ4 Bam(ter)2⁻ (CCG GAT CCT TAT TTC ATG CAA TCT TC; SEQ ID NO:7), and Vδ6L-XhoI (CTC GAG ATG CCT CCT CAC AGC CTG TTC TGT G; SEQ ID NO:8) with 3′ Cδ BSP⁻ (listed above; SEQ ID NO:4).

For the Vγ4/Vδ5 TCR—Vγ4L-EcoRI (GAA TTC CAG ACC ATG AAG AAC CCT GG; SEQ ID NO:9) with Cγ4 Bam (ter) (listed above; SEQ ID NO:7), and Vδ5-XhoI (CTC GAG GGA AGG ATG ATT CTT GCC GC; SEQ ID NO: 10) with 3′ Cδ BSP (listed above; SEQ ID NO:4).

The soluble TCR DNA constructs were then co-transfected into the Sf9 moth cell line along with baculovirus helper DNA (BaculoGold, Pharmingen Corp., or Bacvector 3000, Novagen) to generate a baculovirus-containing culture supernatant that produces soluble TCR molecules, as has been previously described (Kappler et al., Proc Natl Acad Sci USA 91:8462-8466 (1994)). A soluble αβ TCR-producing baculovirus (the DO-11.10 TCR; gift of John Kappler, National Jewish) containing a similar BirA site linked to the C-terminus of the β chain (Lang et al., Science 291:1537-1540 (2001)) was prepared in a similar manner in the inventors' laboratory as a negative control. More specifically, soluble TCR/baculovirus-containing culture supernatants were produced in High Five insect cells, using a multiplicity of infection of approximately 5-10 infectious units per insect cell. The cells were then cultured for 6 days, at 27° C. for the first day, then at 19° C. for the remainder. Culture supernatants containing soluble γδ TCRs were then purified by passing them over anti-Cδ (GL3) sepharose affinity columns. For the soluble αβ control TCR, the culture supernatant was similarly purified by passage over an anti-Cβ (H57-597) column. These columns were prepared using CNBr-activated sepharose CL-4B beads (Sigma, St. Louis, Mo.) in accordance with the manufacturer's directions. However, to pre-clear the supernatant and obtain cleaner preparations, we first passed those supernatants containing soluble γδ TCRs over an anti-Cβ column, and those containing the soluble αβ TCR over an anti-Cδ column, passing the flow-through immediately afterwards over the correct affinity column for the TCR type. Next, the affinity columns were washed with about 25 column volumes of 50 mM NaCl/100 mM Tris pH7.4, and the bound molecules eluted with 50 mM diethylamine in distilled water, pH 11.5. Fractions of 0.9 ml, up to a total of about 10 ml, were serially collected into tubes containing 0.1 ml 1M Tris pH 6.5, to neutralize them. Fractions containing the eluted protein were identified by optical density at 280 nM, combined, and dialyzed overnight to PBS. The products were then concentrated with Centricon-30 units or Amicon Ultra filter devices (Amicon Bioseparations, Millipore, Beverley, Mass.), and stored for up to several months at 4° C. Average yields differed for the various TCRs; the Vγ6/Vδ1 TCR averaged about 0.4 μg/ml of supernatant, whereas the Vγ5/Vδ1 TCR gave a slightly higher yield of about 0.7 μg/ml, the Vγ1/Vδ6.3 and Vγ4/Vδ5 soluble TCRs about 0.3 μg/ml, and the αβ soluble TCR about 1 μg/ml. The TCRs produced each had unique molecular weights that matched those predicted from their primary sequence, after accounting for N-linked glycosylation sites.

In experiments in which tetramers were used, these were then specifically biotinylated using the Bir A enzyme. For some experiments, TcRs with or without biotin were then further purified over a Superdex 200 sizing column by FPLC (AP Biotech), as indicated in the figure legend. Tetramers were generated by treatment with streptavidin, or streptavidin conjugated to the fluorochrome phycoerythrin.

Assessment of Ability of Purified sTcRs to Bind n-zAbs.

Anti-TCR mAbs were purified by passage over protein A or protein G sepharose columns (AP Biotech), concentrated by vacuum dialysis, then dialysed to PBS. To determine that each retained native conformation sufficient to bind to anti-TCR mAbs recognizing native structure, a competition assay was carried out. For each test, 40 ng per sample of anti-TCR mAb was incubated for 10-20 minutes alone or together with sTcRs, in 96 well plates. The diluted mAb or mixture was then transferred into wells of a 96-well flat-bottom plate containing 10⁵ cells of a T cell hybridoma with a known TCR. Cloned T cell hybridomas used include. Binding of the soluble TCR of each hybridoma to the mAb was then determined by a reduction in the presence of the sTcR of mAb available to stain the Vγ5/Vδ1⁺ cells, using a fluorescently labeled secondary antibody (goat anti-rat IG or rabbit anti-hamster Ig, Jackson laboratories, Me.). The mAbs tested in this analysis include the hamster mAbs GL3 (anti-Cδ) and F536 (anti-Vγ5 ), and the rat mAbs KJ1 (DO-11.10 anti-idiotype) and 17C (anti-Vδ6.3).

Flow Cytometry.

Infection with Listeria monocytogenes was used to induce inflammation and a coincident Vγ/Vδ1 cell response by injecting C57BL/10 mice with 2-4×10³ L. monocytogenes i.v., as previously described (Roark et al., J Immunol 156:2214-2220 (1996)). Mice treated with sTcR were given a second i.v. injection 10-30 minutes later, on the other side of the tail vein. Livers and spleens were removed 5 days later for analysis, and the T cells from each purified by nylon wool passage as previously described (Julius et al., Eur J Immunol 3:645-649 (1973); Roark et al., J Immunol 150:4867-4875 (1993)). Liver and spleen γδ T cells were analyzed by flow cytometry using two color analysis, with biotinylated anti-Vγ1 and anti-Vγ4 mAbs plus streptavidin-PE, together with anti-Cδ directly labeled with FITC, as previously described (Mukasa et al., J Immunol 162:4910-4913 (1999)). All staining reagents were purified and conjugated in our own laboratory, using standard techniques. Analysis was carried on a FACSCAN or LSR (Becton Dickenson), using CellQuest software.

Listeria Infection.

Listeria monocytogenes EGD were freshly grown from frozen aliquots in tryptose phosphate broth (Difco Laboratories, Detroit, Mich.) at 37° C. overnight on a shaker. Dilutions of the culture were made in non-pyrogenic PBS, assuming an initial concentration of 2×10⁹/ml. C57BL/10 mice, male or female, bred in-house from Jackson Laboratories stock, of 6 to 12 weeks of age, were injected with 2-3×10³ L. monocytogenes EGD by i.v. injection via the tail vein, in a volume of 0.2 cc in PBS; the exact dose given was confirmed each time by plating dilutions on tryptic soy agar (Difco Laboratories, Detroit, Mich.) plates. Mice were harvested 5 days after inoculation, and spleen and liver T cells prepared for analysis as previously described.

Bacterial Clearance.

C57BL/10 mice were inoculated i.v. into the tail vein with ˜3-4×10⁴ L. monocytogenes, and received 10-30 minutes later sTcR i.v. on the other side of the tail. The remaining bacterial content of spleens and livers was assessed three days later, by plating dilutions of organ homogenates on TSA plates, as previously described (O'Brien et al., J Immunol 165:6472-6479 (2000)).

Soluble TCRs as Staining Reagents.

Cells (2×10⁵ cells/well in 96-well flat bottom culture plates) were incubated for 20 min. at 4° C. with 2.4G2 culture supernatant to block Fcγ receptors, then washed once with staining buffer (BSS containing 2% FBS plus 0.1% sodium azide) and incubated with 1-4 μg/well of soluble TCR, for 60 minutes at 4° C. Cells were washed three times and incubated with either conjugated GL-3 mAb (to detect γδ soluble TCRs) or with conjugated H57-597 mAb (to detect the αβ soluble TCR) for 20 minutes at 4° C. In some experiments, unconjugated mAbs were used to detect bound soluble TCRs, followed by FITC-labeled anti-hamster IgG (Jackson ImmunoResearch, West Grove, Pa.) as a secondary reagent. After the final incubation, cells were washed three times and analyzed on a FACSCAN or FACSCalibur flow cytometer using CellQuest software.

Additionally, in some experiments staining was carried out with directly FITC-conjugated soluble TCRs. These were labeled by the same method used for FITC-conjugation of mAbs, using flourescein isothiocyanate Ion Celite (10% FITC) (Sigma Corp., St. Louis, Mo.).

### Example 1

The following example describes the production of soluble T cell receptors (sTcRs).

Constructs for expressing mouse Vγ and Vδ genes representative of particular γδ T cell subsets were generated by truncating each TCR cDNA just downstream of the cysteine codon in each used to form the γ-δ interchain disulfide bond, and expressed in a baculovirus system. The δ gene of each construct was also modified by the addition of a site specific for the Bir A enzyme of E. coli, such that a biotin group for tetramerization could be added, after the method of Altman et al. (Altman et al., Science 274:94-96 (1996)). The sTcRs used in this study included a canonical Vγ6/Vδ1 TCR, a canonical Vγ5/Vδ1 TCR (closely related to the Vγ6/Vδ1 in having an identical δ chain and Jγ-Cγ, but an unrelated Vγ), and a Vγ1/Vδ6.3 TCR (derived from the hybridoma BNT-19.8). A soluble αβ TCR (derived from the OVA/IEd-reactive hybridoma DO-11.10 (Kappler et al., Proc Natl Acad Sci USA 91:8462-8466 (1994)) was also prepared for comparison. These products were purified by passage over anti-Cδ (or anti-Cβ for the αβ TCR) affinity columns, and eluted with DEA, pH 10.8. The product obtained showed a fairly high degree of purity when analyzed by SDS-PAGE, and the TcRs had the predicted molecular mass both before and after reduction (data not shown). Moreover, the sTcRs prepared in this way appeared to be undegraded and fairly homogenous. N-terminal sequence analysis of the γ and δ chain was also carried out for an earlier version of the Vγ6/Vδ1 sTcR, and this verified that products were the Vγ6 and Vδ1 chains (Roark, C. E. 1995. A study on murine liver γδ T lymphocytes, University of Colorado Health Sciences Center, Dennison Library Ph.D. thesis, 88-94). Most experiments described herein were carried out with sTcRs at this level of purity; all results were also verified with TcRs that were first biotinylated, passed over an FPLC sizing column to get additional purity, and tetramerized before using them, as indicated.

### Example 2

The following experiment describes the verification of native conformation of sTcRs.

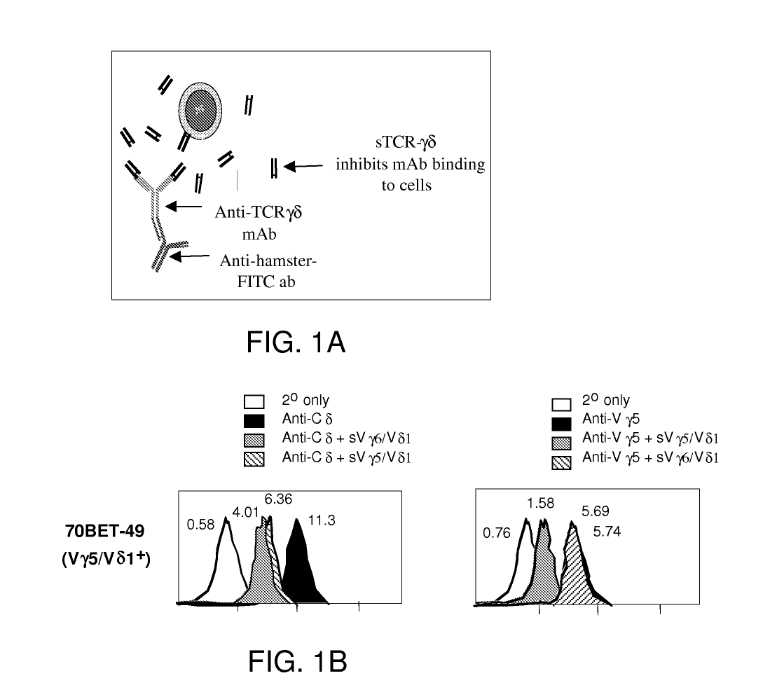

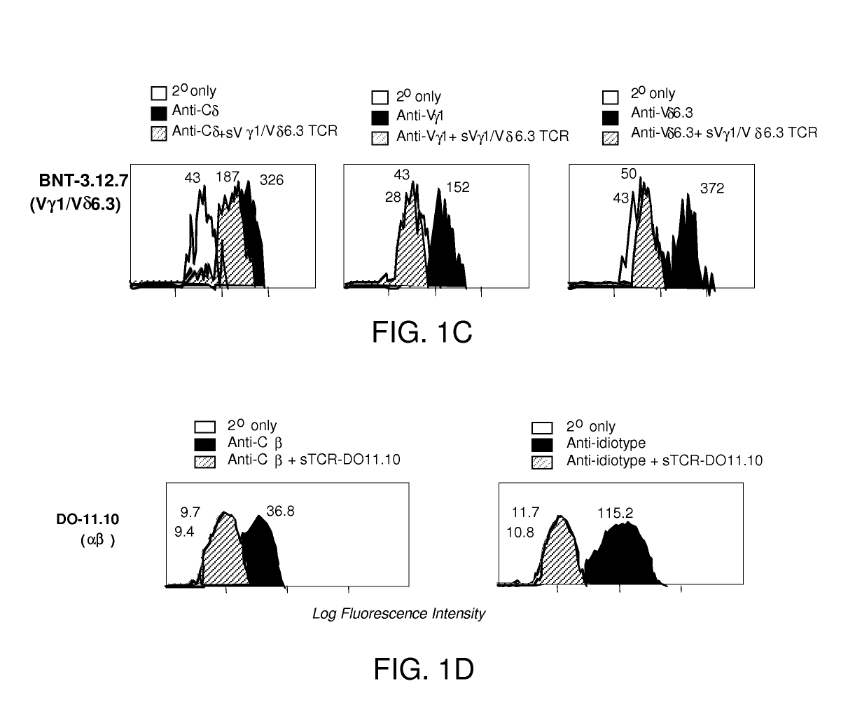

The integrity of the purified Vγ6/Vδ1 and Vγ5/Vδ1 sTcRs was examined by testing whether they retain the ability to bind to anti-TCR mAbs recognizing native structures (Goodman et al., Immunogenetics 35:65-68 (1992); Goodnow et al., Nature 352:532-536 (1991); Havran et al., Proc Natl Acad Sci USA 86:4185-4189 (1989)). Here, a competition assay was used to assess whether sTcR added to a solution containing anti-TCR mAb could reduce the amount of anti-TCR consequently available to stain a TCR⁺ cell line as illustrated in FIG. 1A. Retention of the ability of a sTcR to bind a mAb is thus shown by a reduction in staining with the mAb plus a fluorescently labeled secondary antibody.

Both the Vγ6/Vδ1 sTcR and the Vγ5/Vδ1 sTcR bound to anti-Cδ mAb, but only the Vγ5/Vδ1 sTcR, as expected, also bound to the anti-Vγ5 mAb (FIG. 1B). The sVγ1/Vδ6.3 and sTcR-αβ were also capable of binding to both anti-constant region as well as anti-V-region mAbs (FIGS. 1C and 1D). These results suggest that both the constant and variable regions of the sVγ5/Vδ1, sVγ1/Vδ6.3 and sTcR-αβ are therefore correctly folded into their native configuration. No monoclonal antibodies specific for the Vγ6 and/or Vδ1 region were available to similarly test binding to the variable portion of the Vγ6/Vδ1 sTcR.

### Example 3

This example shows that sVγ6/Vδ1 TCR specifically blocks expansion of the Vγ6/Vδ1⁺ subset in vivo.

Responses of Vγ6/Vδ1 γδ T cells have been reported in a number of different disease models in rodents, including infectious disease models (Ikebe et al., Immunol 102:94-102 (2001); Matsuzaki et al., Eur J Immunol 29:3877-3886 (1999)), an autoimmune model (Mukasa et al., J Immunol 162:4910-4913 (1999); Mukasa et al., J Immunol 159:5787-5794 (1997)), and a model of drug-induced inflammatory damage (Ando et al., J Immunol 167:3740-3045 (2001)). The present inventors' laboratory found several years ago a preferential response of this γδ T cell subset in the livers of C57BL/10 mice infected with Listeria (Roark et al., J Immunol 156:2214-2220 (1996)), the model the inventors used to assess responses of the Vγ6/Vδ1 subset for this study.

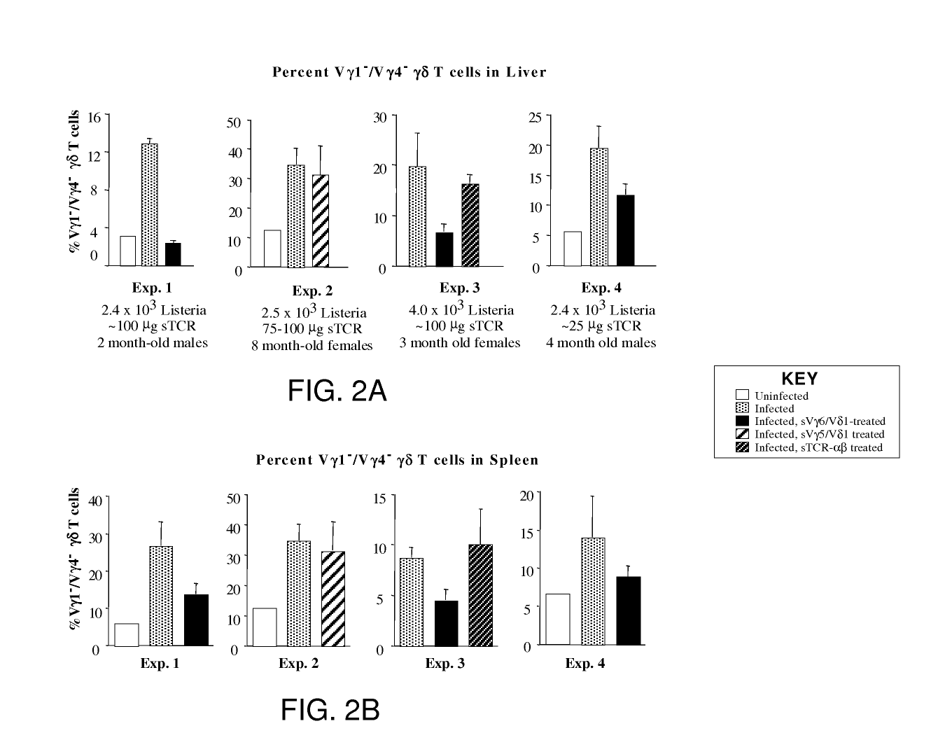

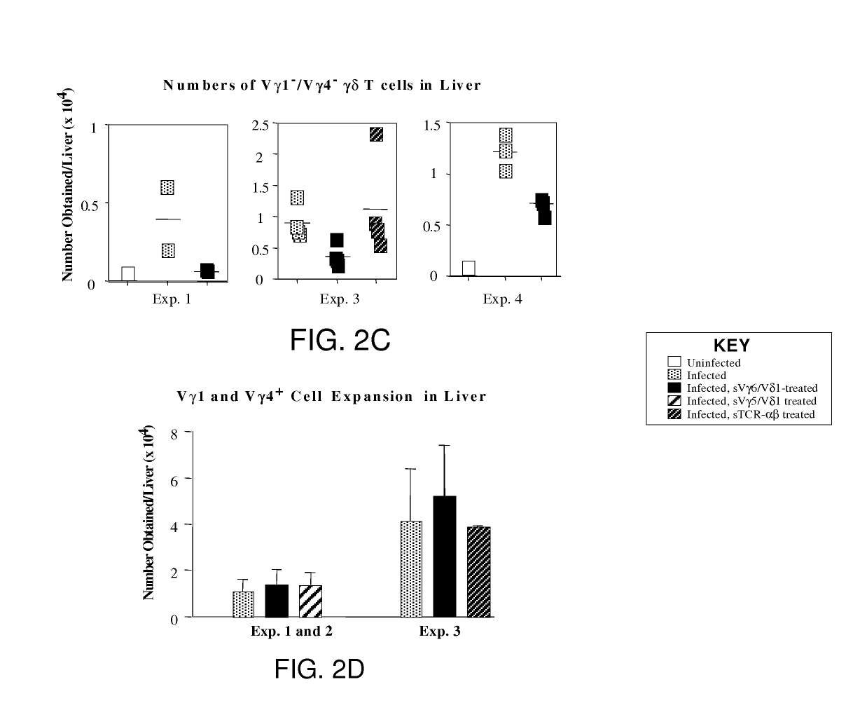

It was reasoned that if sVγ6/Vδ1 TCR could be provided in sufficient quantity in the responding mice, and if this TCR's affinity for its natural ligand was high enough, the response of the Vγ6/Vδ1⁺ cells would be inhibited as a result of competition by the sTcR for ligand binding. To assess the role of the TCR in Vγ6/Vδ1⁺ γδ T cell expansion, mice were treated with a dose of sVγ6/Vδ1 TCR at the time of infection with Listeria (FIG. 2). Because of the lack of any specific mAb for this subset, the level of Vγ6/Vδ1⁺ cells cannot be directly determined by flow cytometry, and must be assessed in other ways, such as by hybridoma analysis (Roark et al., J Immunol 156:2214-2220 (1996)) or by PCR amplification of specific mRNAs (Roark, 1996, ibid.). However, the levels of Vγ6/Vδ1⁺ cells can also be monitored indirectly by flow cytometry (Mukasa et al., J Immunol 162:4910-4913 (1999)) by determining the proportion of γδ T cells staining with anti-Cδ but not with anti-Vγ1 or -Vγ4 mAbs (data not shown). These two mAbs together stain 85-95% of γδ T cells normally present in spleen, lymph node, blood, and liver of C57BL/10 mice).

FIG. 2A shows results from 4 experiments in which the approximate percentage of Vγ6/Vδ1⁺ cells was determined in this way, using C57BL/10 mice of both sexes and of various ages. As can be seen, the percentage of the Vγ1⁻/Vγ4⁻ liver γδ T cells (mainly Vγ6⁺) increased 3-5 fold during infection. This increase that was not affected in mice treated with Vγ5/Vδ1 sTcR or sTcR-αβ, was absent in mice receiving Vγ6/Vδ1 sTcR. This suggested that the expansion of Vγ6/Vδ1⁺ cells was selectively blocked by the presence of Vγ6/Vδ1 sTcR, but not other sTcRs. In experiment 4 of FIG. 2A, mice were given a dose of only about 25 μg of sVγ6/Vδ1 TCR. This was not as effective a dose as the ˜100 μg used in the other experiments, although it still reduced Vγ6/Vδ1⁺ γδ T cell expansion compared to the untreated control.

The expansion of Vγ6⁺ cells (Vγ1/Vγ4⁻ cells) in the spleens of the same animals was also investigated. The inventors had previously found that, when using lower doses of Listeria (˜4×10²/mouse i.v.) in a Listeria-sensitive mouse strain, an increase of 2-3 fold in splenic Vγ1⁻/Vγ4⁻ cells is often seen during infection (O'Brien et al., J Immunol 165:6472-6479 (2000) and unpublished observations). The inventors hypothesized suspected that these cells are also Vγ6/Vδ1⁺. As shown in FIG. 2B, the expansion of splenic Vγ1⁻/Vγ4⁻ cells was also largely blocked by sVγ6/Vδ1 treatment, but not by treatment with sVγ5/Vδ1 or sTcR-αβ. Thus, the Vγ1⁻/Vγ4⁻ cells that expand in infected spleen also appear to be largely composed of Vγ6/Vδ1⁺ cells.

Because T cells of all types expand in the spleen and liver of mice infected with a low dose of Listeria, the degree to which the expansion of the Vγ6/Vδ1 subset was blocked could not be determined by percentage. Therefore, the numbers of these T cells that were actually obtained from each liver during listeriosis was calculated. The numbers of liver Vγ1⁻/Vγ4⁻ cells obtained from mice treated with sVγ6/Vδ1 TCR was in fact on the average near the basal level found in the uninfected controls for experiments in which 100 μg of sVγ6/Vδ1 TCR was used (exp. 1 and 3 in FIG. 2C). When only ˜25 μg of this sTcR was used, although the numbers of Vγ1⁻/Vγ4⁻ indicated some increase, fewer Vγ1⁻/Vγ4⁻ cells were obtained than in untreated infected controls. The numbers of Vγ1⁺ or Vγ4⁺ liver γδ T cells in the same animals in contrast were unaffected by sVγ5/Vδ1 or sTcR-αβ treatment (FIG. 2D); this provides an internal specificity control showing that while sVγ6/Vδ1 treatment blocks Vγ6/Vδ1 cell expansion, it does not affect the expansion of other γδ T cells. It therefore appears a dose of ˜100 μg of this sTcR is sufficient to completely prevent the expansion of the Vγ6/Vδ1 subset, whereas a dose of ˜25 μg reduces the expansion by about 50% (FIG. 2C).

The use of an indirect method of detecting Vγ6/Vδ1⁺ cells in the above experiments might lead to false conclusions if the treatment with sVγ6/Vδ1 actually was instead or as well influencing the level of another Vγ1⁻/Vγ4⁻ γδ T cell population. However, this is not likely in view of the fact that the expanded Vγ1⁻/Vγ4⁻ population does not stain with anti-Vγ1 and −Vγ4 mAbs, and also fails to stain with specific mAb for Vγ5 or Vγ7 (data not shown), thereby ruling out four of the six functional mouse Vγ chains; other than Vγ6, the only other possibility is Vγ2, which is extremely rare. However, to directly confirm that cells expressing the Vγ6/Vδ1 TCR were indeed reduced in infected mice after sVγ6/Vδ1 treatment, northern blots using Vγ6 and Vδ1 probes were also carried out, using whole cell RNA purified from liver T cells. Vγ6 and Vδ1 levels in infected mice treated with sVγ6/Vδ1 TCR were much lower than those in untreated mice (˜4-fold), and in fact were near the level present in uninfected liver (data not shown).

### Example 4

The following example shows that treatment with the sVγ6/Vδ1 TCR improves clearance of Listeria in infected mice.

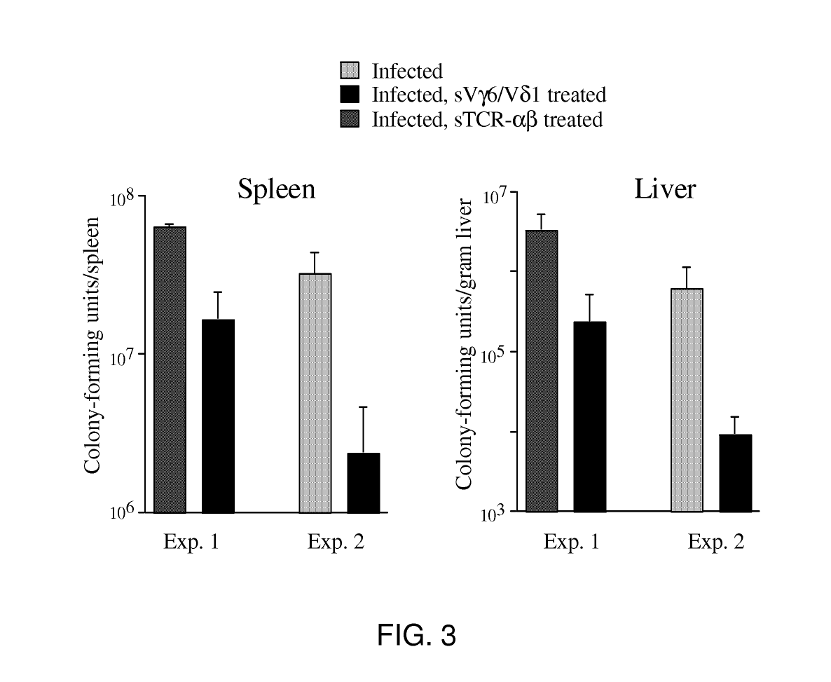

The inventors next tested whether inhibiting the expansion of Vγ6/Vδ1 γδ T cells had any consequence on disease outcome. The inventors previously found that Vγ1⁺ γδ T cells have a negative effect on bacterial clearance early in infection in the C57BL/10 mouse strain (O'Brien et al., J Immunol 165:6472-6479 (2000)). However, the C57BL/10 strain is too efficient at clearing Listeria to examine this with the low Listeria doses that were used to measure the expansion of Vγ6/Vδ1 cells. Therefore bacterial clearance effects were tested using an increased Listeria dose (˜1/10 LD₅₀), and at an earlier time point (day 3 instead of day 5), although the apoptotic effect of Listeria on lymphocytes (Merrick et al., Am. J. Pathol. 151:785-792 (1997)) does not allow the visualization of Vγ6/Vδ1 expansion under these conditions. Again, providing sTcRs at the same time as the infectious agent, particularly in liver, but to a lesser degree also in spleen, mice which received the sVγ6/Vδ1 TCR showed decreased numbers of bacteria, compared to those treated with the irrelevant αβ sTcR (FIG. 3). This improvement in bacterial clearance following blockage of a γδ T cell subset response, similar to that seen previously following depletion of the Vγ1⁺ subset but considerably stronger, ranges from an average difference of 16-fold in experiment 1 to over 60-fold in experiment 2 (in which a slightly lower dose of Listeria was used).

The larger effect seen in liver vs. spleen may reflect the greater expansion of this subset in liver than in spleen (FIG. 2B). Many studies have shown that the inflammatory response is largely responsible for the clearance of Listeria in mice during the first few days of infection (e.g., see Bancroft et al., J. Immunol. 139:1104-1107 (1987); Dunn et al., Infect. Immun. 59:2892-2900 (1991); Conlan et al., J. Exp. Med. 179:259-268 (1994); Czuprynski et al., J. Immunol. 152:1836-1846 (1994)). Therefore, assuming that the sVγ6/Vδ1 TCR is blocking the activation of the Vγ6/Vδ1 subset by binding to an induced TCR ligand, the reduced bacterial counts imply that the Vγ6/Vδ1⁺ cells have an overall anti-inflammatory effect during early listeriosis. This experiment represents the first direct examination of the function of the Vγ6/Vδ1 γδ T cell subset, because of the lack of a mAb specific for this TCR. It also demonstrates that the functions of the Vγ6/Vδ1⁺ subset are elicited via the TCR, presumably following interaction with a ligand.

### Example 5

The following example describes the use of a soluble γδ T cell receptor to characterize the natural ligand.

Because the Vγ6/Vδ1⁺ population responds during inflammation, the inventors have postulated that the ligand for the Vγ6/Vδ1 TCR is a host-produced molecule induced on the surface of some cells in response to inflammatory signals. Therefore, it was thought that it might be possible to detect this ligand using a soluble version of the Vγ6/Vδ1 TCR as a staining reagent. As a test of the feasibility of this approach, a soluble version of a γδ TCR (KN6) whose ligand has already been identified was first generated and was shown to selectively identify its ligand (data not shown). Given the success with this test γδ TCR, the inventors had evidence that other soluble γδ TCRs could be used similarly. Under conditions similar to those used for the KN6 TCR, the inventors were in fact able to detect staining with the Vγ6/Vδ1 soluble TCR on a number of cell lines. All cells that stained typically showed a positive peak together with a negative or very low-staining peak (data not shown). In general, neither T nor B lymphocyte-derived cell lines stained with the Vγ6/Vδ1 soluble TCR, but keratinocyte, fibroblast, and epithelial cell lines generally stained well, suggesting that the natural ligand for the Vγ6/Vδ1 TCR is normally not expressed by lymphocytes but instead by other cell types. Consistently, when using a directly-fluoresceinated version of the Vγ6/Vδ1 TCR to examine normal cells, it was determined that nylon wool purified T cells from the liver or spleen were largely negative, whereas other cells, particularly common in the liver, stained brightly with this TCR (data not shown). When liver cells were examined from mice with an ongoing Listeria infection, the T cells still failed to stain, but other cells now showed enhanced staining with the Vγ6/Vδ1 TCR. This observation is consistent with a need for enhanced expression of the ligand in order for the Vγ6/Vδ1⁺ subset to expand.

As a control, some of the cell lines were also stained with other soluble TCRs, including the Vγ5/Vδ1 canonical TCR, the KN6-derived Vγ4/Vδ5 TCR, a Vγ1/Vδ6.3 TCR derived from the autoreactive hybridoma BNT-19.8, and an αβ TCR derived from the ovalbumin/Ia^{d}-reactive hybridoma DO-11.10. Results showed that some staining above background was also evident with the soluble αβ TCR (about a 4-fold increase in mean fluorescence), though it was comparatively weaker than that seen with the Vγ6/Vδ1 soluble TCR (about an 8-fold increase in mean fluorescence). The Vγ5/Vδ1 soluble TCR showed weak but consistent staining of virtually every cell line stained by the Vγ6/Vδ1 TCR. The Vγ1/Vδ6.3 TCR also stained many of the same cell lines as did the Vγ6/Vδ1 soluble TCR, although the staining was usually weaker, and the pattern of staining often appeared to be different (data not shown). Finally, as mentioned above, some cell lines, including the XB-2 cell line, also stained very strongly with the Vγ4/Vδ5 KN6-derived soluble TCR. It therefore appears that the Vγ4/Vδ5 TCR and the Vγ6/Vδ1 TCR may detect different molecules on the XB-2 cell line.

To test for the specificity of the binding of the soluble TCRs, a cold-competition experiment was performed using an excess of an unlabeled soluble TCR, in an attempt to block the binding of the soluble Vγ6/Vδ1 TCR to the XB-2 cell line. Results showed that after pre-incubating the XB-2 cells with a twenty-fold excess of αβ TCR, the subsequent degree of staining with the soluble Vγ6/Vδ1 TCR was virtually undiminished. In contrast, when unlabeled soluble Vγ6/Vδ1 TCR was used to block a directly labeled version of the same TCR, the degree of staining of the bright peak was reduced by about one third.

Using the XB-2 line as a representative of a Vγ6/Vδ1 ligand-bearing cell, the inventors have attempted to induce a higher expression of the ligand in various ways, including treating XB-2 and some of the other cell lines with LPS, subjecting them to heat shock, and depriving them of fetal bovine serum in their culture medium. None of these treatments had any evident effect. Conversely, the inventors have attempted to treat ligand-bearing cells in a number of ways that might denature or destroy the ligand. In one experiment, XB-2 cells were treated with different enzymes that are compatible with live cells. All three proteases used reduced the staining to some degree, with pronase and trypsin treatment almost eliminating the staining altogether. In contrast, neuraminidase treatment of the XB-2 cells had no evident effect. As a control, to rule out the possibility that residual protease in the treated cells had simply destroyed the soluble Vγ6/Vδ1 TCR so that it was no longer available to stain the cells, the soluble Vγ6/Vδ1 TCR-containing supernatant used to stain the treated cells was saved and re-incubated it with fresh, untreated XB-2 cells. The transferred supernatant stained untreated XB-2 cells at a level that was undiminished as compared to fresh soluble Vγ6/Vδ1 TCR, thus ruling out this possibility (data not shown).

The soluble version of the Vγ6/Vδ1 canonical TCR was also used as a staining reagent to directly detect what appears to be a ligand for this TCR. To establish the feasibility of this approach, a soluble Vγ5/Vδ4 TCR derived from the hybridoma KN6 was first tested for its ability to stain its ligand, the T22^{b} molecule, a non-classical MHC class I. In order to get detectable staining with the KN6 TCR, it was necessary to use about twenty times more of the TCR (on a molar basis) than would ordinarily be used if the reagent had been a high-affinity monoclonal antibody. This was to be expected, based on the previously reported affinity of the KN6 TCR for its ligand, which lies in the low-affinity antibody range. Using a T22^{b} transfectant and an untransfected version of the same cell line, staining with the KN6-derived γδ TCR was demonstrated, and the intensity of the staining correlated with that obtained with an anti-T22^{b} mAb. The parent transfectant did not stain, nor did other soluble γδ TCRs stain the T22^{b} transfectant. However, the parent line of this transfectant, T2, is a human T cell line. When a number of mouse cell lines were similarly tested, although none stained with the anti-T22^{b} mAb, several stained with the soluble KN6 TCR, in some cases quite strongly (data not shown). Because the KN6 TCR is also known to recognize a related class Ib molecule, T10^{b}, it seemed likely that other ligands might exist for this TCR. Although T10^{b} itself cannot explain the staining of these mouse cell lines because the anti-T22^{b} mAb also detects T10^{b}, other candidate class Ib molecules might, such as the one encoded by the closely-related BALB/c-derived T9^{c} gene.

Next, a directly labeled Vγ6/Vδ1 TCR was used in an attempt to track the Vγ6/Vδ1 TCR's ligand during a Listeria infection. Many non-T cells in the normal liver stained quite brightly with this soluble TCR, although only weak staining has been detected on T cells. The brightly staining cells are probably hepatocytes based on their high frequency in liver, although this has not yet been directly shown; in the spleen, brightly staining cells were much more rare. After infection with Listeria, the bright liver cells only showed about a 30% enhancement in staining. However, the liver cells were tested on day 5 of the infection, when the Vγ6/Vδ1⁺ cells were expected to be present at peak levels, and this might be considerably beyond the time that the ligand is maximally expressed. Alternatively or as well, the activation of these cells during inflammation/infection could require other cofactors or cytokines which must be present along with the TCR ligand in order to bring about activation of this subset. In fact, the Vγ6/Vδ1 subset appears to express Toll-like receptor 2 when induced during E. coli infection, a molecule which could act as a second signal for these cells.

The inventors found that other soluble γδ TCRs were also able to stain certain of the cell lines that were tested, some quite brightly, and even the αβ control soluble TCR showed low-level staining on many cell lines (data not shown). Nonetheless, for several reasons and without being bound by theory, the present inventors believe that the Vγ6/Vδ1 soluble TCR binding that has been observed is specific. First, in a cold competition experiment, whereas unlabeled Vγ6/Vδ1 TCR was able to compete with a labeled version of itself for binding, the αβ soluble TCR was ineffective in competing with the Vγ6/Vδ1 soluble TCR for binding. Second, cells in the liver that stain with the Vγ6/Vδ1 TCR showed enhanced staining during infection with Listeria, coincident with the marked expansion of Vγ6/Vδ1⁺ cells at this site. Third, a number of cells and cell lines, in particular B and T lymphocytes, failed to stain with the Vγ6/Vδ1 soluble TCR to any measurable extent, whereas others stained quite brightly. The staining of some cell lines with two of the other soluble γδ TCRs, the Vγ1/Vδ6.3 TCR and the Vγ4/Vδ5 KN6-derived TCR, because it is restricted to only some cells and is very bright, may also be due to the binding of a specific ligand.

The molecular nature of the cell surface ligand detected by the soluble Vγ6/Vδ1 TCR remains unresolved. Because all cells that stained typically showed a positive peak together with a negative or very low-staining peak whose relative percentages varied from experiment to experiment, the ligand may be expressed only at certain points during the cell cycle. Treatment of a cell line staining brightly with the Vγ6/Vδ1 soluble TCR with three different proteases completely or partially abrogated their ability to stain with this TCR, whereas neuraminidase had no effect, suggesting that the ligand is a cell surface protein molecule. However, it is possible that the chemical nature of the ligand is in fact non-protein, but that it in some way depends upon a cell surface protein for expression. For instance, the ligand could be a glycosylation product present on certain proteins, whose expression is induced. Alternatively, the ligand could be a complex of molecules, one or more of which is a cell surface protein.

All references cited herein are incorporated herein by reference in their entireties.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to regulate a γδ T cell-mediated immune response in a mammal, comprising administering to said mammal a soluble γδ T cell receptor.

2. The method of claim 1, wherein said soluble γδ T cell receptor comprises a single γ chain and a single δ chain linked by a disulfide bond.

3. The method of claim 1, wherein said soluble γδ T cell receptor is a multimer of soluble γδ T-cell receptors comprising γ chains and δ chains linked by disulfide bonds.

4. The method of claim 1, wherein said soluble γδ T cell receptor comprises a murine Vγ1 chain, a human Vγ9 chain, or the equivalent thereof.

5. The method of claim 1, wherein said soluble γδ T cell receptor comprises a murine Vγ4 chain, a human Vγ9 chain, a human Vγ8 chain, or the equivalent thereof.

6. The method of claim 1, wherein said soluble γδ T cell receptor comprises a murine Vγ6 chain, a human Vγ9 chain, a human Vγ8 chain, or the equivalent thereof.

7. The method of claim 1, wherein said soluble γδ T cell receptor is administered at a dose of from about 0.01 microgram×kilogram⁻¹ and about 10 milligram×kilogram⁻¹ body weight of said mammal.

8. The method of claim 1, wherein said soluble γδ T cell receptor is administered at a dose of from about 0.1 microgram×kilogram⁻¹ and about 10 milligram×kilograms⁻¹ body weight of said mammal.

9. The method of claim 1, wherein the step of administering said soluble γδ T-cell receptor is by a route selected from the group consisting of: aerosol, topical, intratracheal, transdermal, subcutaneous, intradermal, intravenous, intramuscular, intraperitoneal and direct injection to a tissue.

10. The method of claim 1, wherein said mammal has or is at risk of developing an intestinal condition.

11. The method of claim 10, wherein said intestinal condition is selected from the group consisting of Crohn's disease, ischemic colitis, irritable bowel disease, and colon cancer.

12. The method of claim 1, wherein said mammal has or is at risk of developing a lung condition associated with inflammation.

13. The method of claim 12, wherein said lung condition is selected from the group consisting of airway hyperresponsiveness, pneumonia, tuberculosis, and a primary or metastatic lung tumor.

14. The method of claim 1, wherein said mammal has or is at risk of developing a skin condition associated with inflammation.

15. The method of claim 14, wherein said skin condition is selected from the group consisting of: a skin lesion caused by bacterial infection, viral infection or laceration, and a skin cancer.

16. The method of claim 1, wherein said mammal has or is at risk of developing a condition associated with inflammation of the reproductive tract.

17. The method of claim 16, wherein said condition is selected from the group consisting of: infection caused by bacterial or viral infection that involve the epithelial mucosal lining, a tubal infection, preventing tubal factor infertility, and a cancer selected from the group consisting of ovarian cancer, cervical cancer, uterine cancer, prostate cancer and testicular cancer.

18. The method of claim 1, wherein said mammal has or is at risk of developing inflammation caused by a γδ T cell subset, and wherein the soluble γδ T cell receptor is a soluble T cell receptor expressed by said γδ T cell subset.

19. The method of claim 18, wherein said soluble γδ T cell receptor comprises a murine Vγ6 chain and a murine Vδ1 chain, a human Vγ8 or Vγ9 chain and a human Vδ2 chain, or the equivalent receptor thereof.

20. The method of claim 1, wherein said mammal has or is at risk of developing myocarditis caused by a γδ T cell subset, and wherein the soluble γδ T cell receptor is a soluble T cell receptor expressed by said γδ T cell subset.

21. The method of claim 20, wherein said soluble γδ T cell receptor comprises a murine Vγ4 chain, a human Vγ9 chain, a human Vγ8 chain or the equivalent receptor thereof.

22. The method of claim 20, wherein administration of said soluble γδ T cell receptor increases the activity of a γδ T cell subset expressing a murine Vγ1⁺ T cell receptor, a human Vγ9⁺ T cell receptor, or the equivalent receptor thereof.

23. The method of claim 1, wherein said mammal has or is at risk of developing an infection with Listeria monocytogenes, wherein said soluble γδ T cell receptor comprises a murine Vγ1 chain, a murine Vγ6 chain, a human Vγ9 chain, a human Vγ8 chain, or the equivalent thereof, and wherein administration of said soluble γδ T cell receptor increases clearance of Listeria monocytogenes from said mammal.

24. The method of claim 1, wherein said mammal has or is at risk of developing airway hyperresponsiveness caused by inflammation, and wherein said soluble γδ T cell receptor does not comprise a murine Vγ4 chain, a human Vγ9 chain, or the equivalent thereof, wherein administration of said soluble γδ T cell receptor results in an increase in the activity of a γδ T cell subset that expresses said murine Vγ4, said human Vγ9, or the equivalent thereof so that airway hyperresponsiveness is reduced in said mammal.

25. The method of claim 1, wherein said mammal is a human.

26. A composition for regulating a γδ T cell-mediated immune response in a mammal, comprising:
a) a soluble γδ T cell receptor; and
b) an agent that regulates inflammation in said mammal.
